(19) United States
(12) Patent Application Publication
(10) Pub. No.: US 2024/0189822 A1
(43) Pub. Date: Jun. 13, 2024

(54) THREE-DIMENSIONAL MICROFLUIDIC METASTASIS ARRAY

(71) Applicant: Regents of the University of Minnesota, Minneapolis, MN (US)

(72) Inventors: Elizabeth Crist, Wayzata, MN (US); David Wood, Minneapolis, MN (US); Michael C. McAlpine, Minneapolis, MN (US); Ruitao Su, Allston, MA (US)

(21) Appl. No.: 18/556,529

(22) PCT Filed: Apr. 21, 2022
(86) PCT No.: PCT/US2022/071843

§ 371 Date: Oct. 20, 2023

Related U.S. Application Data
(60) Provisional application No. 63/201,276, filed on Apr. 21, 2021.

(51) Int. Cl. B01L 3/00; B33Y 80/00; C12M 1/12; C12M 3/00; C12M 3/06

(52) U.S. Cl. CPC B01L 3/502761; B33Y 80/00; C12M 21/08; C12M 23/16; C12M 25/04; B01L 2200/16; B01L 2300/0654; B01L 2300/087; B01L 2300/0874

(57) ABSTRACT

The describes example systems, devices, and techniques. In one example, a device includes a body extending away from a substrate, which includes a first end with an open-facing port configured to allow introduction of a tissue sample, and a second end that forms an open outlet proximal the major surface of the substrate. At least a portion of the body includes therein a tissue chamber for the tissue sample. At least one microfluidic channel on the major surface of the substrate is fluidly connected to the tissue chamber, and includes an inlet upstream of the tissue chamber and an outlet downstream of the tissue chamber. A separation element is between the tissue chamber and the at least one microfluidic channel. The tissue chamber, the separation element and the microfluidic channel occupy a single layer on the substrate.

This application is a PCT application claiming priority to U.S. Provisional Patent Application No. 63/201,276, filed Apr. 21, 2021, the entire contents of which is incorporated herein by reference.

### STATEMENT REGARDING FEDERALLY SPONSORED RESEARCH AND DEVELOPMENT

This invention was made with government support under Grant Nos. CA235385 and CA210190 awarded by the National Institutes of Health. The government has certain rights in the invention.

### BACKGROUND

Anticancer drug development requires extensive time, money, and resources to bring new pharmaceutical agents to the market. Despite advancements in biomedical research and technology, out of hundreds of thousands of potential anticancer drug candidates that enter the pipeline, only about 5% succeed in clinical trials. A major hurdle in the drug discovery process is the lack of pre-clinical and pre-animal in vitro assays that closely mimic human disease and enable more biologically relevant metrics to quantify drug efficacy.

Recent advances in bioengineered three-dimensional (3D) in vitro cancer models have significantly reduced the cost, labor and time associated with in vivo animal testing. Additionally, these humanized 3D models overcome the challenge of species-to-species variability, which hinders translatability of findings obtained in animal models. However, the majority of existing in vitro platforms used to study cancer are highly reductionist and are incapable of evaluating higher order functions in cancer progression, such as transendothelial migration and metastasis. Moreover, these existing assays predominantly rely on suspensions of single cancer cells to model tumors, which fail to capture the complex heterogeneity and organization within tumor microenvironments.

In commercial drug testing settings, the majority of in vitro assays are two-dimensional and do not accurately model factors such as, 3D human tumor biology, dynamic vessel perfusion, barriers to cancer drug resistance, and secondary sites for metastasis. For example, some in vitro assays are configured to study growth and migration of tumor cell aggregates or organoids, but lack the capability to assess the impact of drugs administered through biophysiological blood vessels to larger and more biologically relevant multicellular tumor spheroids (MCTS), patient-derived xenograft (PDX)-derived organoids, and primary tumor biopsies, as these tumor samples exceed dimensions capable of inclusion in existing assays. Additionally, existing assays are not configured to evaluate metastatic dissemination of malignant cells through vascularized multicellular bodies to downstream tissue sites.

In one example, the metastatic spread of cancer cells in the human body from a primary lesion to a secondary site is comprised of several dynamic processes. The metastatic cascade can be broadly described by local invasion, intravasation, circulation, extravasation and colonization. Cancer cell migration as single cells or multicellular units is integral to metastatic progression and is controlled by numerous genetic and environmental factors from the surrounding tissue microenvironment (TME). The prevalence of single cell and multicellular migration throughout the course of metastasis remains to be determined, and elucidation of the link between migration mode and distinct steps of the metastatic cascade will be imperative to identifying therapeutic agents that inhibit stage-dependent dissemination.

Current in vitro models of metastasis do not recapitulate distinct physical barriers intrinsic to the metastatic cascade and enable visualization of cancer cell migration propagated throughout each process. Existing bioengineered cancer models use reductionist approaches to study a subset of events in metastasis and primarily model tumors using single cancer cell suspensions. While these assays provide insight into genetic and environmental factors that contribute to cancer cell migration in breast cancer, assessments of how multimodal migration propagates throughout metastasis has not been possible. Humanized 3D in vitro models of metastasis are needed that recapitulate all stages of metastasis, utilize a tumor model with greater physiologic relevance, and enable manipulation and quantification of factors that regulate cancer cell migration and metastasis.

### SUMMARY

A simple and inexpensive 3D in vitro assay model is needed that can accurately model biologically relevant metrics to rapidly assess cancer therapeutics and monitor cancer cell dissemination throughout the stages of metastasis. Such an in vitro assay can be used to maximize the probability of therapeutic success in clinical trials and help predict early failures in the cancer drug development pipeline.

Described herein are 3D assay devices that can be used to screen for agents that have an effect on living tumors generated from MCTS, PDX-derived organoids, or primary tumor biopsies. For example, the device can be used to screen for anti-angiogenesis agents, anti-metastasis agents, wound healing agents and tissue engineering agents. In one aspect, the assay is a device that can be used to form a high throughput tumor microenvironment for investigations of multi-cellular interactions (e.g., high throughput anti-cancer drug screening), for drug discovery, and for personalized medicine. For example, the tumor microenvironment can be used as an in-vitro test for new drugs that may help stop the spread of cancer in the human body in a research and commercial setting, and to match the best anti-cancer drug to a particular patient in a hospital setting.

In an example, the devices of the present disclosure provide 3D in vitro assays that form at least one tumor microenvironment (TME) that is fluidically connected to endothelialized microfluidic channels and at least one secondary downstream tissue site for metastasis. The TME is configured to, for example, study the effect of anti-cancer therapeutics on, or evaluate the metastasis of, at least one MCTS, PDX-derived organoid, or primary tumor biopsy, which include multiple cell types, and in some cases can be vascularized. Compared to the suspension of single tumor cells and tumor cell aggregates used in some in vitro assays, the shrinkage or growth and inhibition of metastasis of large MCTS, PDX-derived organoids, or primary tumor biopsies can provide a more physiologically accurate model to evaluate barriers to tumor drug resistance, as well as the efficacy of anti-cancer therapeutics. In one example, the assays of the present disclosure provide a metastasis mimetic platform with an engineered microfluidic vascular network that can be used to model pathophysiologic tumors and recapitulate each stage of the metastatic cascade, which can be used to investigate tumor heterogeneity and components of the TME that influence cancer cell migration and the metastatic dissemination of cancer cells.

In one example, the present disclosure is directed to a device that includes a body extending away from a substrate, the body including: a first end with an open-facing port configured to allow introduction of a tissue sample, and a second end of the body opposite the first end, wherein the second end forms an open outlet proximal the major surface of the substrate, and wherein at least a portion of the body includes therein a tissue chamber for the tissue sample; at least one microfluidic channel on the major surface of the substrate, wherein the microfluidic channel is fluidly connected to the tissue chamber, and wherein the microfluidic channel includes an inlet upstream of the tissue chamber and an outlet downstream of the tissue chamber; and separation element between the tissue chamber and the at least one microfluidic channel. The tissue chamber, the separation element and the microfluidic channel occupy a single layer on the substrate.

In another example, the present disclosure is directed to a device including a substrate with a major surface; a body extending away from the major surface of the substrate, wherein the body includes: a first end with an open-facing port configured to allow introduction into the tissue chamber of a tissue sample chosen from multicellular tumor spheroids derived from established cell lines, engineered animal models, patient-derived xenografts, or primary tumor biopsies, wherein the tissue sample has a diameter of greater than about 100 microns, and an open second end proximal the major surface of the substrate; a primary tissue chamber between the first end of the body and the second end of the body; at least one microfluidic channel on the major surface of the substrate, wherein the microfluidic channel underlies the body, and wherein the microfluidic channel includes an inlet upstream of the tissue chamber and an outlet downstream of the tissue chamber; a membrane between the primary tissue chamber and the at least one microfluidic channel; and a secondary tissue chamber fluidly connected to the primary tissue chamber, wherein the secondary tissue chamber includes at least one portion on a first side of the microfluidic channel and a second portion on a second side of the microfluidic channel.

In another example, the present disclosure is directed to a device, including: a microfluidic channel; a body with a first compartment on a first side of the microfluidic channel and a second compartment on a second side of the microfluidic channel, wherein the first compartment includes a tissue chamber with an open port configured to allow introduction of a tissue sample chosen from multicellular tumor spheroids derived from established cell lines, engineered animal models, patient-derived xenografts, or primary tumor biopsies, wherein the tissue sample has a diameter of greater than about 100 microns, and wherein the first compartment and the second compartment are fluidly connected to the microfluidic channel; a media reservoir comprising a media inlet and a media outlet, wherein the media inlet and the media outlet are fluidly connected to the microfluidic channel; and a removable plug in the microfluidic channel, wherein the removable plug controls flow of a media composition from the media reservoir into the microfluidic channel.

In another example, the present disclosure is directed to an in vitro assay system, including: a substrate; a body extending away from a major surface of the substrate, wherein the body has an open first end sized to allow introduction of a tissue sample chosen from multicellular tumor spheroids derived from established cell lines, engineered animal models, patient-derived xenografts, or primary tumor biopsies, wherein the tissue sample has a diameter of greater than about 100 microns and an open second end opposite the first end, wherein the body includes therein a tissue chamber, an extracellular matrix material in at least a portion of the tissue chamber; at least one microfluidic channel on the major surface of the substrate, wherein the at least one microfluidic channel includes a media inlet upstream of the tissue chamber and a media outlet downstream of the tissue chamber; a separation element between the tissue chamber and the microfluidic channel, wherein the separation element is configured to retain the extracellular matrix material in the tissue chamber, and wherein the separation element includes at least one passage containing the extracellular matrix material; and an interface region downstream of the at least one passage in the separation element, wherein the interface region provides fluid communication between the extracellular matrix material and a media composition in the at least one microfluidic channel.

In another example, the present disclosure is directed to a method for making a three-dimensional in vitro assay, the method including: extruding through a nozzle an elongate polymeric base filament in a pattern on a surface of a flexible substrate; moving the nozzle to stepwise extrude and stack a plurality of polymeric filaments onto the base filament such that each of the polymeric filaments extruded onto the base filament contact one another along their lengths to form a device, the device including: a body extending away from a major surface of the substrate, the body having an open top port and an open bottom opposite the top port, wherein the open top port is sized to allow introduction into the body of a tissue sample chosen from multicellular tumor spheroids derived from established cell lines, engineered animal models, patient-derived xenografts, or primary tumor biopsies, wherein the tissue sample has a diameter of greater than about 100 microns, a primary tissue chamber in the body between the open top port and the open bottom thereof, at least one microfluidic channel on the major surface of the substrate wherein the at least one microfluidic channel is fluidly connected to the primary tissue chamber; and a separation element between the primary tissue chamber and the microfluidic channel, wherein the separation element is configured to retain an extracellular matrix material in the primary tissue chamber; and at least partially curing polymeric filaments to form a self-supporting arrangement of structures on the surface of the substrate.

In another example, the present disclosure is directed to a method for modeling tumor development, the method including: in a tumor microenvironment including: substrate; a body on a major surface of the substrate, the body having an open first end and an open second end opposite the first end, wherein the body includes a tissue chamber between the open first end and the open second end thereof, and wherein the tissue chamber includes an extracellular matrix material; at least one microfluidic channel on the major surface of the substrate, wherein the microfluidic channel includes an inner surface with a layer of endothelial cells, and wherein the microfluidic channel has an inner diameter of about 200 microns to about 1 mm; and a separation element between the tissue chamber and the microfluidic channel, wherein the separation element is configured to retain a tissue sample n the well, and wherein the separation element includes at least one passage containing the extracellular matrix material; and a media inlet upstream of the tissue chamber and a media outlet downstream of the tissue chamber; inserting a tissue sample into the tissue chamber, wherein the tissue sample is chosen from multicellular tumor spheroids derived from established cell lines, engineered animal models, patient-derived xenografts, or primary tumor biopsies, and wherein the tissue sample has a diameter of greater than about 100 micros in one example or about 500 microns in other examples; injecting a media composition into the media inlet; and forming an interface region downstream of the separation element, wherein the interface region provides fluid communication between extracellular matrix material and the media composition in the at least one microfluidic channel.

In another example, the present disclosure is directed to a method including: three-dimensionally (3D) printing a device including: a body extending away from the substrate, wherein a first end of the body includes an open-facing port configured to allow introduction of a tissue sample chosen from multicellular tumor spheroids derived from established cell lines, engineered animal models, patient-derived xenografts, or primary tumor biopsies, wherein the tissue sample has a diameter of greater than about 500 microns, and a second end of the body opposite the first end forms an open outlet proximal the major surface of the substrate, and wherein at least a portion of the body has therein a primary tissue chamber; at least one microfluidic channel on the major surface of the substrate, wherein the microfluidic channel is fluidly connected to the tissue chamber, and wherein the microfluidic channel includes an inlet upstream of the tissue chamber and an outlet downstream of the tissue chamber; and a separation element between the primary tissue chamber and the at least one microfluidic channel; and wherein the tissue chamber, the separation element and the microfluidic channel occupy a single layer on the substrate.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a schematic overhead view of an example of a microfluidic device according to the present disclosure.

FIG. 2A is a schematic overhead view of an example of a microfluidic device according to the present disclosure including a plurality of tissue chambers in a series arrangement.

FIG. 2B is a schematic overhead view of an example of a microfluidic device according to the present disclosure including a plurality of tissue chambers in a series and parallel arrangement.

FIG. 2C is a schematic overhead view of an example of a microfluidic device according to the present disclosure including a plurality of tissue chambers in a series and parallel arrangement.

FIG. 2D is a schematic overhead view of an example of a microfluidic device according to the present disclosure including dual media inlets and a plurality of tissue chambers in a series and parallel arrangement.

FIG. 2E is a schematic overhead view of an example of a microfluidic device according to the present disclosure including a plurality of tissue chambers in a series arrangement.

FIG. 2F is a schematic overhead view of an example of a microfluidic device according to the present disclosure including a plurality of tissue chambers in a series arrangement.

FIG. 2G is a schematic overhead view of an example of a microfluidic device according to the present disclosure including a plurality of tissue chambers in a series arrangement.

FIG. 2H is a schematic overhead view of an example of a microfluidic device according to the present disclosure including a plurality of rectangular tissue chambers in a series arrangement.

FIG. 2I is a schematic overhead view of an example of a microfluidic device according to the present disclosure including a single tissue chamber.

FIG. 2J is a schematic overhead view of an example of a microfluidic device according to the present disclosure including a single tissue chamber and a plurality of vascularization channels in parallel.

FIG. 2K is a schematic overhead view of an example of a microfluidic device according to the present disclosure including a plurality of tissue chambers.

FIG. 2L is a schematic overhead view of an example of a microfluidic device according to the present disclosure including a plurality of tissue chambers.

FIG. 2M is a schematic overhead view of an example of a microfluidic device according to the present disclosure including a plurality of tissue chambers.

FIG. 2N is a schematic overhead view of an example of a microfluidic device according to the present disclosure including a plurality of tissue chambers and respective support channels.

FIG. 3A is an overhead perspective view of an example of a device of the present disclosure fabricated using 3D printing or additive manufacturing.

FIG. 3B is a magnified overhead view of the device of FIG. 4A.

FIG. 4A is a schematic representation of a system and method for 3D printing a microfluidic channel.

FIG. 4B is a 3D model of a self-supporting 3D printed structure including a fluid passage with a triangular cross-sectional shape.

FIG. 4C is a 3D model of a self-supporting 3D printed structure including a fluid passage with a circular cross-sectional shape.

FIG. 4D is a 3D model of a self-supporting 3D printed structure with a hexagonal domed shape.

FIG. 4E is a 3D model of a self-supporting 3D printed structure with a conical domed shape.

FIGS. 5A-5B are plots of a bending moment analysis of a self-supporting wall printed with a straight profile.

FIGS. 5C-5E are composite cross-sectional images of silicone walls of varying incline angles and an overhang length of 700 μm, with scale bars of 200 μm.

6A-6D are photographs of 3D printed microfluidic channels and chambers with walls cut open to display the cross-sectional profiles, with 1 mm scale bars.

FIGS. 6E-6F are scanning electron microscope (SEM) images of triangular and circular channels, respectively with a width of about 100 μm. The photographs have scale bars of 100 μm.

FIG. 7 is a schematic representation of an example of a 3D printed microfluidic valve construction.

FIG. 8 is a flow chart of an example of a process for making a 3D printed microfluidic device according to the present disclosure.

FIG. 9A is a schematic overhead view of an example of a microfluidic according to the present disclosure.

FIG. 9B is a schematic side view of the device of FIG. 9A.

FIG. 9C is a magnified cross-sectional view along lines A-A of the device of FIG. 9A.

FIG. 10A is a photograph of an overhead view of an example of a microfluidic device according to the present disclosure.

FIG. 10B is a photograph of a side view of the microfluidic device of FIG. 10A.

FIG. 10C is a photograph of an overhead view of an example of a microfluidic device according to the present disclosure.

FIG. 10D is a photograph of a side view of the microfluidic device of FIG. 10C.

FIG. 11A is a photograph of a monoculture multicellular tumor spheroid (MCTS) of Example 1 composed of MDA-MB-231-GFP cells.

FIG. 11B is a photograph of a monoculture multicellular tumor spheroid (MCTS) of Example 1 composed of Hs578T-GFP cells.

FIG. 11C is a photograph of a monoculture multicellular tumor spheroid (MCTS) of Example 1 composed of BT-549-GFP cells.

FIG. 11D is a photograph of a monoculture multicellular tumor spheroid (MCTS) of Example 1 composed of MCF7-GFP cells.

FIG. 11E is a photograph of a monoculture multicellular tumor spheroid (MCTS) of Example 1 composed of MCF-10A-mRuby cells.

FIG. 11F is a plot of the average diameter of 10000 cells of the MCTS of FIGS. 11A-11E at day 5.

FIG. 12A is a photograph of a co-culture tumor spheroid including both cancer cells and normal cells of Example 1 formed by mixing MDA-MB-231-GFP cells and MCF-10A-mRuby cells at a ratio of 1:1.

FIG. 12B is a photograph of a co-culture tumor spheroid including both cancer cells and normal cells of Example 1 formed by mixing MDA-MB-231-GFP cells and MCF-10A-mRuby cells at a ratio of 1:2.

FIG. 12C is a photograph of a co-culture tumor spheroid including both cancer cells and normal cells of Example 1 formed by mixing MDA-MB-231-GFP cells and MCF-10A-mRuby cells at a ratio of 1:4.

FIG. 12D is a photograph of a co-culture tumor spheroid including both cancer cells and normal cells of Example 1 formed by mixing MDA-MB-231-GFP cells and MCF-10A-mRuby cells at a ratio of 1:9.

FIG. 12E is a photograph of a co-culture tumor spheroid including both cancer cells and normal cells of Example 1 formed by mixing Hs578T-GFP cells with MCF-10A-mRuby cells at a ratio of 1:4.

FIG. 12F is a photograph of a co-culture tumor spheroid including both cancer cells and normal cells of Example 1 formed by mixing Hs578T-GFP cells with MCF-10A-mRuby cells at a ratio of 1:9.

FIG. 12G is a plot of the average diameter of 10000 cells of the MCTS of FIGS. 12A-12F at day 5.

FIG. 13A is a photograph of an overhead view of a MCTS in the tissue chamber of the microfluidic device of Example 1 taken at day 1.

FIG. 13B is a magnified view of the photograph of FIG. 13A.

FIG. 13C is a photograph of an overhead view of a MCTS in the tissue chamber of the microfluidic device of Example 1 taken at day 3.

FIG. 13D is a magnified view of the photograph of FIG. 13C.

FIG. 13E is a photograph of an overhead view of a MCTS in the tissue chamber of the microfluidic device of Example 1 taken at day 5.

FIG. 13F is a magnified view of the photograph of FIG. 13E.

FIG. 13G is a photograph of an overhead view of a MCTS in the tissue chamber of the microfluidic device of Example 1 taken at day 7.

FIG. 13H is a magnified view of the photograph of FIG. 13G.

FIG. 14A is a photograph of an overhead view of the tissue chamber of the device of Example 1 showing the interface region at day 11.

FIG. 14B is a magnified view of the photograph of FIG. 14A.

FIG. 14C is a photograph of an overhead view of the tissue chamber of the device of Example 1 showing the interface region at day 13.

FIG. 14D is a magnified view of the photograph of FIG. 14C.

FIG. 14E is a photograph of an overhead view of the tissue chamber of the device of Example 1 showing the interface region at day 14.

FIG. 14F is a magnified view of the photograph of FIG. 14E.

FIG. 14G is a photograph of an overhead view of the tissue chamber of the device of Example 1 showing the interface region at day 16.

FIG. 14H is a magnified view of the photograph of FIG. 14G.

FIG. 15A is a plot of tumor area over time according to Example 1 for Hs578T-GFP cells and Hs578T-GFP cells mixed with MCF-10A-mRuby cells at a ratio of 1:4.

FIG. 15B is a plot of tumor invasion area over time according to Example 1 for Hs578T-GFP cells.

FIG. 15C is a plot of tumor invasion area over time according to Example 1 for the Hs578T-GFP cells mixed with MCF-10A-mRuby cells at a ratio of 1:4.

Like symbols in the drawings indicate like elements.

### DETAILED DESCRIPTION

In general, the present disclosure is directed to microfluidic devices that can be used as a 3D bioassay, e.g., for drug screening, personalized medicine, tissue engineering, wound healing, and other applications. The devices include a tissue chamber with an open-facing port configured to contain a biologically relevant tumor such as, for example, a multicellular tumor spheroid (MCTS), a patient-derived xenograft (PDX) derived organoid, or a primary tumor biopsy with a diameter of at least about 500 microns, in some examples (or a diameter of at least about 100 microns in other examples). The port allows rapid placement of whole tissue samples, such as biopsied cancer or human tissue samples in their native configuration into the tissue chamber with, for example, a biopsy punch.

The tissue chamber can be filled with a biologically relevant extracellular matrix material to form a tumor microenvironment. In some examples, the extracellular matrix material is maintained in the tissue chamber by a separation element including an arrangement of posts. The extracellular matrix material forms an interface with at least one microfluidic channel, which can in some examples be plated with cells such as endothelial cells. The microfluidic channels provide inlets and outlets for drugs or biological material to be introduced or expelled from the tumor microenvironment. The growth, intravasation, extravasation, and metastasis initiated from the multicellular tumor spheroid, PDX-derived organoid, or primary tumor biopsy and other stromal cells in the tumor microenvironment can then be observed over time to assess the efficacy of anti-cancer spreading drug discovery applications, anti-cancer growth drug discovery, tissue engineering and wound healing drug discovery, identifying chemoattractive and/or chemorepulsive agents, and matching the correct dose and combination of above drugs to a patient, among other applications. In this manner, the devices and techniques described herein may be configured for assessing biologic and cell therapies, such as T cell and Natural Killer (NK) cell therapies because the therapy can be delivered through a blood vessel and to a primary tissue site. For example, the therapy in question can be applied to a sample tissue in a tissue chamber of a microfluidic device and examine the effect of that therapy on the sample tissue (e.g., tumor evolution and/or metastasis) in the tissue chamber.

For example, to fit the best drug to an individual patient, a tissue biopsy of the patient's tumor can be placed in the tissue chamber of the tumor microenvironment, with either a culture of the patient's endothelial cells or cells from an endothelial cell line. Different anti-angiogenesis, anti-metastasis, and other anti-cancer drugs may also be input into the microenvironment, and the tumor in the tissue chamber observed to determine the extent to which the individual's cancer responds to the drugs and the extent to which metastasis is inhibited.

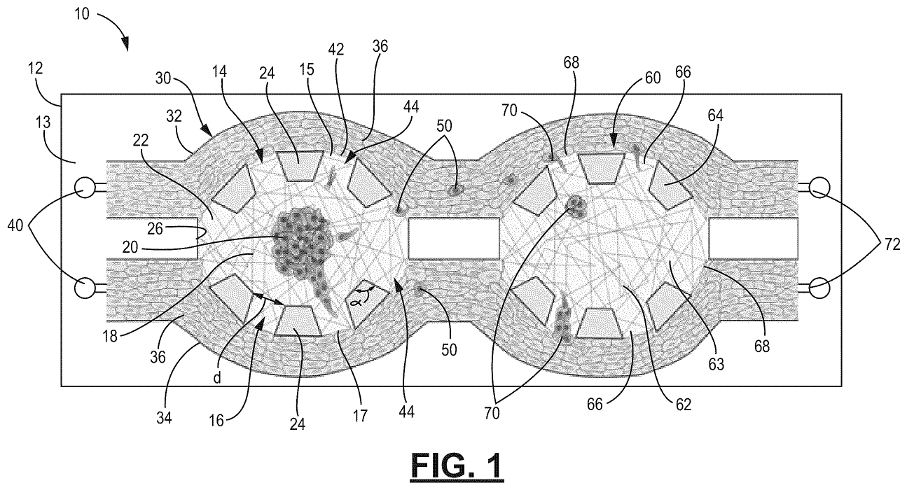

Referring now to FIG. 1, a schematic illustration (which is not to scale) of a 3D assay device 10 includes a substrate 12 with a major surface 13, which may be substantially flat, or may be irregular or include curvature. In some examples, the substrate 12 may be optically clear or substantially transparent to visible light, which can facilitate observation or analysis of tumors or other cells in the device with microscopy or other analytical tools. Suitable substrate materials include, but are not limited to, metals, polymers, plastic, and glass. Examples of suitable polymers for the substrate 12 include polydimethylsiloxane (PDMS), poly(methyl methacrylate) (PMMA), polystyrene (PS), SU-8, and cyclic olefin copolymer (COC). In particular examples, all or a portion of the substrate 12 and other portions of the device are made of a biocompatible material, e.g., the material is compatible with, or not toxic or injurious to, living material (e.g., cells, tissue).

The assay device 10 includes a body 14 extending away from the surface 13 of the substrate 12. In some examples, which are not intended to be limiting, the body can have a height of about 1 mm to about 5 mm above the surface 13. In various examples, the body 14 is made from any of the same biocompatible materials as the substrate 12, and in some examples is a flexible polymeric material such as polydimethylsiloxane (PDMS), poly(methyl methacrylate) (PMMA), polystyrene (PS), SU-8, and cyclic olefin copolymer (COC). The body may be formed by any suitable technique including, for example, injection molding, soft photolithography, 3D printing, or combinations thereof.

The body 14 includes a first end 15 with an open-facing port 16. The open-facing port 16 is configured to allow introduction of large tumor spheroids or other tissue samples into the body 14, and in some examples is sufficiently large to allow passage of tissue samples with a diameter of greater than about 100 microns, greater than about 500 microns, greater than about 1 mm, or greater than about 1.5 mm, or greater than about 2 mm. As noted above, the large open-facing port 16 makes possible the insertion of a large tissue sample in its native configuration with a biopsy punch.

The body 14 further includes an open second end 17 opposite the open-facing port 16. The open second end 17 of the body 14 allows the contents of the body to be in fluid communication with other microfluidic channels on the surface 14 of the substrate 12. In the present application the term microfluidic channel refers to regions, channels and/or chambers for the flow and/or containment of fluids and/or gels, typically on microliter, nanoliter, or picoliter scale.

An interior region of the body 14 includes a tissue chamber 18 between the open-facing port 16 and the open second end 17. The tissue chamber 18 includes dimensions selected to contain a particular tumor or tissue sample for analysis. The dimensions of the tissue chamber may vary widely, and in some examples the tissue chamber has an inside diameter of about 50 microns to about 1 mm, or about 200 microns to about 2 mm. The types of tissues suitable for analysis in the tissue chamber 18 can also vary widely, and in some examples can include large multicellular tumor spheroids (MCTS) 20, organoids, or biopsies having a core of dead cells overlain by a layer of highly proliferative malignant cells. In some cases, the tumor samples in the tissue chamber 18 can include vasculature, and the growth of blood vessels toward the MCTS or away from the MCTS can be observed in the tissue chamber 18. In addition to the large tumor sample, the tissue chamber 18 can further be configured to contain other types of human stromal or cancerous organoids, cell clusters, aggregates, individual cells, and mixtures and combinations thereof.

The tissue chamber 18 further includes an extracellular matrix material 22, which forms a 3D scaffold that surrounds and retains the tumor spheroid, organoid, or biopsy 20, and allows the tumor spheroid, organoid, or biopsy 20 to grow and proliferate. The extracellular matrix material 22 also allows other materials input into the tissue chamber 18 to reach the tumor spheroid 20, and for materials expelled from the tumor spheroid 20 to proceed out of the tissue chamber 18 via the open send end 17 of the body 14. Suitable extracellular matrix materials include, but are not limited to, biocompatible gel-like materials such as collagen, fibronectin, and gels available under the trade designation MATRIGEL from Corning Life Sciences, Corning, NY, or CULTREX from R&D Systems, Inc., Minneapolis, MN, and may also include polymeric gels, which in some case may be at least partially curable using ultraviolet (UV) radiation, heat and the like.

The body 14 further includes a separation element, which in this example includes an arrangement of posts 24 between the tissue chamber 18 and the open second end 17. The number of posts 24, as well as the shape, dimensions, and spacing of the posts 24, are selected to retain a selected extracellular matrix material 22 within the tissue chamber 18 during filling, during insertion of the tumor sample 20, and as the tumor sample grows and develops.

In some examples, which are not intended to be limiting, the posts 24 have a width of about 0.1 mm, a length of about 0.1 mm from a wall 26 of the body 14, and a height of about 0.25 mm. The cross-sectional shape of the posts 24 when viewed from above the body 12 can vary widely, and in some examples, can be circular or elliptical, rectangular, square, triangular, trapezoidal, hexagonal, a teardrop, or combinations thereof. The shapes of the posts 24 may be the same or different. In particular aspects, the height-to-width ratio of each post is from about 1 to about 4. The number of posts 24 may also vary widely, and can be about 2 to about 20, or about 4 to about 12.

In various examples, which are not intended to be limiting, the posts 24 are triangular or trapezoidal structures with a tip angle α of about 200 to about 80°, or about 400 to about 60°. In various examples, the distance d between the posts 24 is about 1 micrometer to about 500 micrometers, about 5 micrometers to about 450 micrometers, about 10 micrometers to about 400 micrometers, or about 50 micrometers to about 300 micrometers.

In the example shown in FIG. 1, the body 14 includes 6 cantilevered trapezoidal posts 24 extending inward into the tissue chamber 18 from the circumferential wall 26 of the body 14. The posts 24 taper or converge toward a center of the tumor chamber 28, and include a tip angle α selected to retain a particular type of extracellular matrix material.

The device 10 further includes an arrangement of least one microfluidic channel 30, and in the example of FIG. 1 includes a first microfluidic channel 32 and a second microfluidic channel 34 extending laterally from opposed sides of the body 14. However, in other examples, the microfluidic channel 30 may be oriented below the body 14 and pass under the open second end 17 thereof, or even above the body 14.

In the example of FIG. 1, the microfluidic channels are fluidly connected to the tissue chamber 18, and interface with the open second end 17 of the body 14. In various examples, which are not intended to be limiting, the microfluidic channels 32, 34 have an internal diameter of about 50 microns to about 200 microns, or about 100 microns to about 200 microns. The microfluidic channels 32, 34 can be made from a wide variety of materials, but flexible polymeric materials that mimic human vasculature have been found to provide a more biologically relevant tumor microenvironment 10. In some examples, the microfluidic channels may be made from polymeric materials such as, for example, polydimethylsiloxane (PDMS), poly(methyl methacrylate) (PMMA), polystyrene (PS), SU-8, and cyclic olefin copolymer (COC). The microfluidic channels 32, 34 may be formed by any suitable technique including, for example, injection molding, soft photolithography, 3D printing, additive manufacturing, or combinations thereof.

In some examples, the interior surfaces of the microfluidic channels 32, 34 may be endothelialized, which in this application refers to a substantially continuous layer 36 of interconnected human endothelial cells formed on the interior surfaces of the microfluidic channels 32, 34. In some examples, the layer 36 is about 1 cell thick, which provides a flexible microfluidic channel 32, 34 with properties similar to those of human blood vessels or lymphatic tissues.

The device 10 further includes at least one media inlet 40 upstream of the tissue chamber 18 in the body 14. In some examples, the body 14 optionally includes a media inlet (not shown in FIG. 1) for adding a biological fluid directly into the tissue chamber 18. Of course, media may also be added to the tissue chamber 18 via the open-facing port 16. Biologically useful fluids (not shown in FIG. 1) maybe in pipetted, injected or pumped with a pump from a syringe, a reservoir, or the like into the media inlets 40 and then enter the microfluidic channels 32, 34. In various examples, suitable biological fluids for input into the device 10 include media compositions with a biological agent chosen from cells, cell aggregates, a peptide fragment, a nuclease, a nucleic acid encoding a nuclease, oligo nucleotide, a protein, peptide a DNA editing template, guide RNA, a therapeutic agent (for example, a drug), a plasmid DNA encoding protein, siRNA, monoclonal antibodies, Cas9 mRNA, and mixtures and combinations thereof.

When the media composition flows into the microfluidic channels 32, 34 an interface region 42 forms between the flowing medium and the extracellular matrix material present in the channels 44 between the posts 24. The interface region 42 allows fluid exchange between the tissue chamber 18 and the flowing medium in the microfluidic channels 32, 34. As shown schematically in FIG. 1, components in the tissue chamber 18 such as, for example, cell aggregates 50, migrate through the interface region 42 and merge into the flowing medium in the microfluidic channels 34, 36 downstream of the tissue chamber 18.

In the example of FIG. 1, the device 10 includes an optional secondary body 60 including a secondary tissue chamber 62 with an extracellular matrix material 63. In some examples, as shown in FIG. 1, the optional body 60 extends above the surface 13 of the substrate 12 like the body 14, and may have the same or a different shape, height, and the like.

In FIG. 1, the secondary body 60 includes optional posts 64, which may be the same or different than the posts 24 in the primary tissue chamber 18. As shown in FIG. 1, cells or cell aggregates 70 migrate from the primary tissue chamber 18 through the microfluidic channels 32, 34 and enter the secondary tissue chamber 62 via an interface 68 with the extracellular matrix material 63 formed in passages or channels 66 between the posts 64. In another example (not shown in FIG. 1), the secondary body 60 can include a membrane to provide an interface between the media in the microfluidic channels 34, 36 and the extracellular matrix material 63 in the secondary tissue chamber 62.

Once lodged in the secondary tissue chamber 62, the cells 70 can grow and develop in the secondary tissue chamber 62, thus forming a tumor metastasis model for the tumor spheroid 20 in the primary tissue chamber 18.

The device 10 of FIG. 1 further includes at least one media outlet 72, which may be connected to a reservoir or another arrangement of microfluidic channels (not shown in FIG. 1).

In various examples, which are not intended to be limiting and are provided as an example, a pump, or a pumping system including a combination of pumps, connected to the media inlets 40 may include a syringe pump, a gravity driven flow device, a peristaltic pump, a piston pump, a diaphragm pump, a gear pump, a hydraulic pump, or a combination thereof. The pump or pumping system may provide continuous perfusion of media or other fluids into the media inlet 40, a pulsed perfusion mimicking the pumping pattern of a human heart, or a combination thereof. In some examples, the media or other fluids may be recirculated from the media outlet 72 back to the media inlet 40 to provide a substantially continuous flow loop through the primary tissue chamber 18 and the secondary tissue chamber 62 (if present).

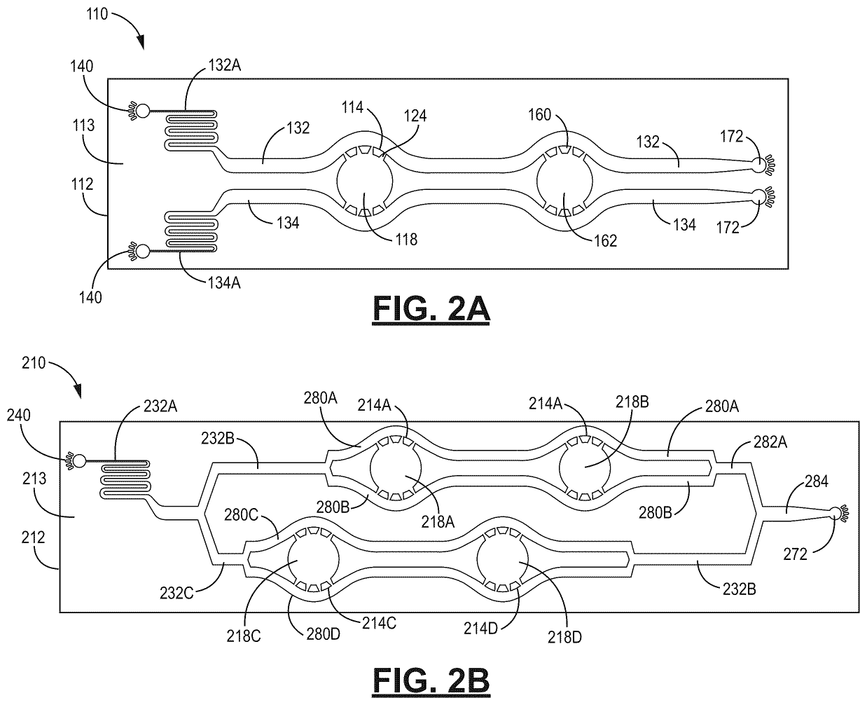

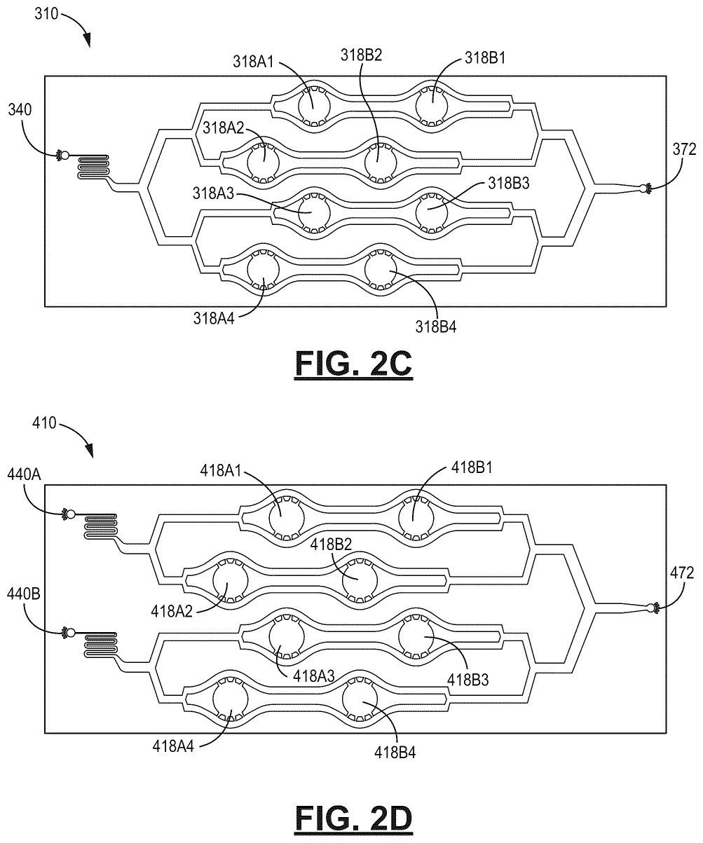

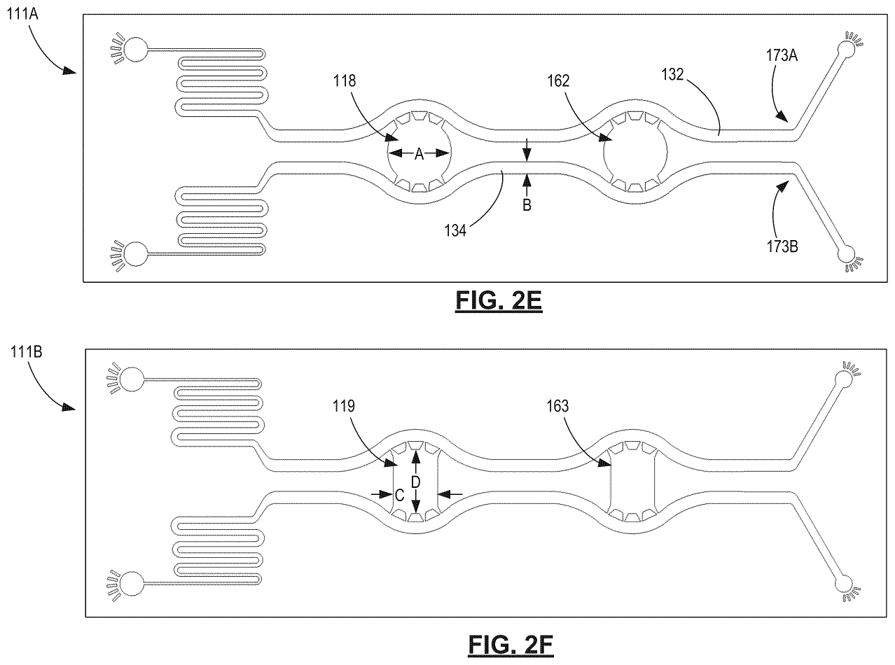

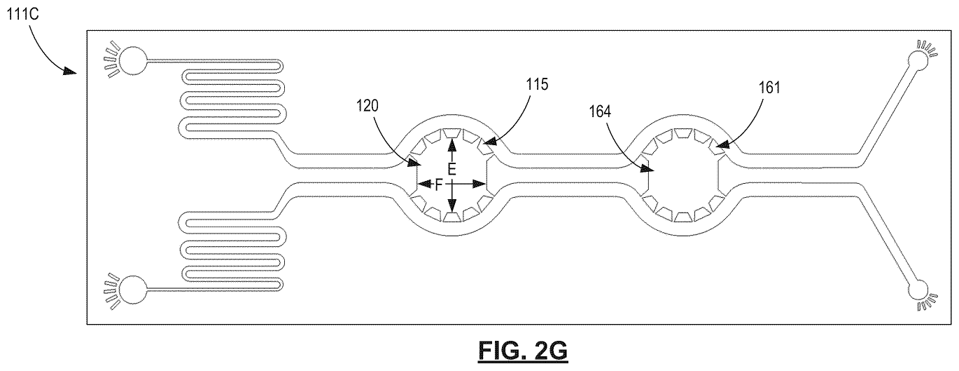

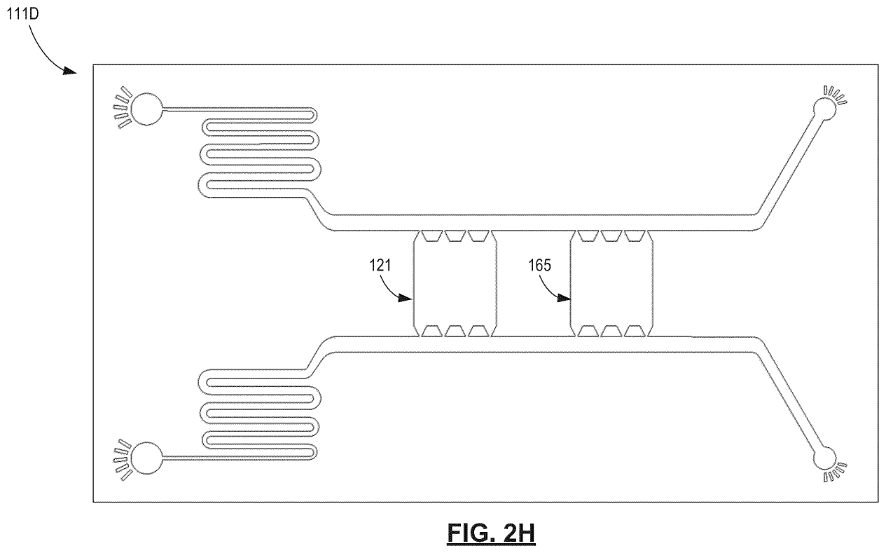

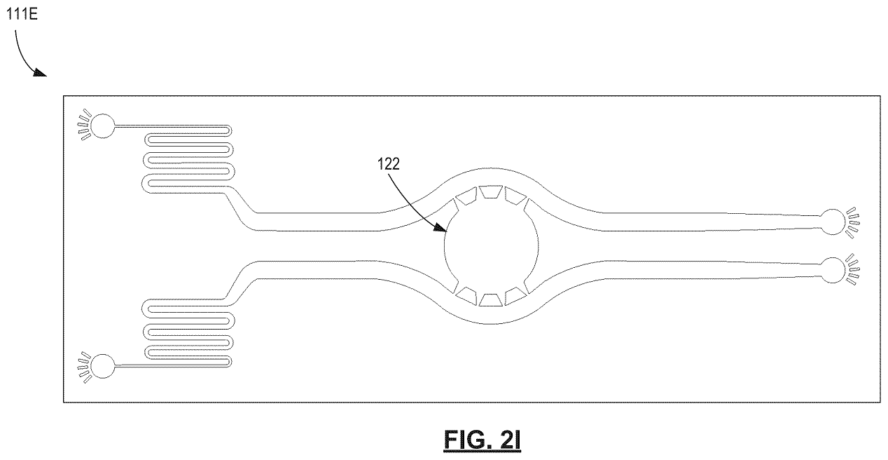

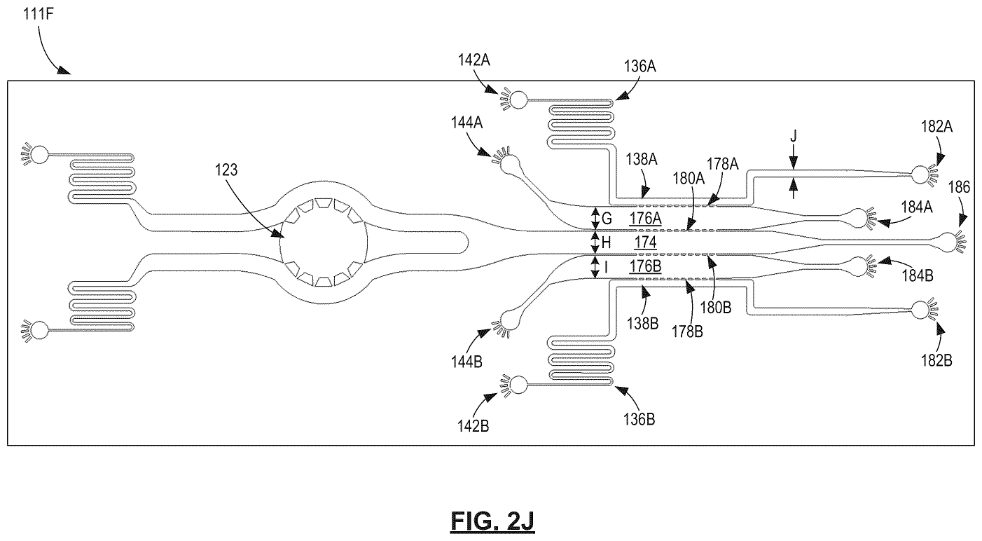

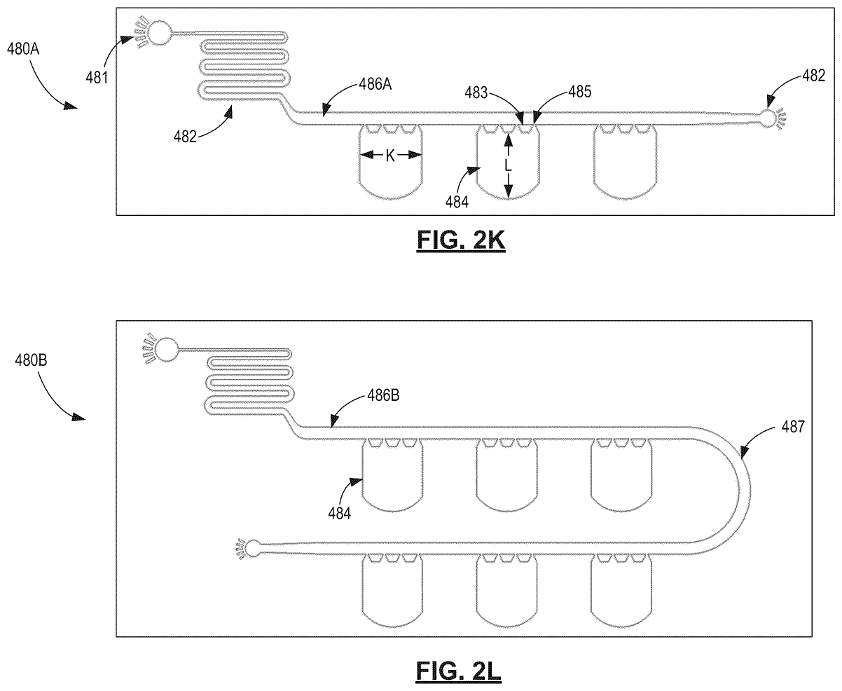

Referring now to FIG. 2A, an overhead view of a microfluidic device 110 is shown on a major surface 113 of a substrate 112. Microfluidic device 110 may be an example of device 10 in FIG. 1. The microenvironment of microfluidic device 110 includes fluid inlets 140 each located upstream of a serpentine portion 132A, 134A of a microfluidic channel 132, 134. Serpentine portions 132A and 134A may provide one or more functions to device 110, such as gradually widening the flow channel in order to reduce flow velocity before reaching tissue chamber 118 and reducing the likelihood that clogs would form or reduce flow because the widening channel dimension would promote continued flow instead of any restriction. The microfluidic channels 132, 134 are positioned laterally of a primary body 114 with a primary tissue chamber 118 and an arrangement of posts that create channels between microfluidic channels 132, 134 and primary tissue chamber 118 between the posts. The microfluidic channels 132, 134 also are positioned laterally with respect to a secondary body 160 including a secondary tissue chamber 162 located downstream of, and in series with, the primary tissue chamber 118. The secondary tissue chamber 162 includes an arrangement of posts that create channels between microfluidic channels 132, 134 and secondary tissue chamber 162 between the posts. Media outlets 172 are located downstream of the series of tissue chambers 118 and 162. While not shown in FIG. 2A, tissue samples may be loaded into tissue chambers, such as tissue chambers 118 and 162, via respective ports disposed above the respective tissue chambers. After the tissue and potentially other ingredients, such as media and/or collagen, the ports may be sealed to external air with one or more covers, such as a coverslip, PDMS sheet, or any other type of cover. In this manner, microfluidic device 110 may include three layers, such as substrate 112, major surface 113, and the cover of the ports for each tissue chamber (not shown).

The flow rate of fluid through any of microfluidic channels described herein may be selected to promote desired cell growth or other activity. In some examples, the flow rate may be chosen to achieve a shear stress on the cells that may be in the range of approximately 0.1 to approximately 10 dynes per square centimeter. This shear stress may be a function of channel cross-sectional and flow rate. For example, a larger diameter channel may require a higher flow rate to achieve a desired shear stress on the cells. As another example, the dimensions of the channels within the device may be chosen to achieve a desired shear stress on the cells given a target flow rate for the device.

In another example shown schematically in FIG. 2B, a microfluidic device 210 is shown on a major surface 213 of a substrate 212. The device 210 includes a fluid inlet 240 located upstream of a serpentine portion 232A of a microfluidic channel, which branches into branch regions 232B, 232C. The branch region 232B is upstream of microfluidic channels 280A, 280B, which link bodies 214A, 214B in series. The bodies 214A, 214B each include a respective tissue chamber 218A, 218B. The branch region 232C is upstream of microfluidic channels 280C, 280D, which link bodies 214C, 214D in series. The bodies 214C, 214D each include a respective tissue chamber 218C, 218D.

The microfluidic channels 280A, 280B merge into a microfluidic channel 282A, and the microfluidic channels 280C, 280D merge into a microfluidic channel 282B, such that the tissue chambers 218A, 218B are arranged in parallel with the tissue chambers 218C, 218D. The microfluidic channels 282A, 282B merge into a microfluidic channel 284, which is upstream of a media outlet 272.

Many different microfluidic device designs are possible, depending on the intended application. For example, as shown in the schematic of FIG. 2C, a microfluidic device 310 includes a fluid inlet 340 and a fluid outlet 372. The device 310 includes four pairs of primary tissue chambers 318A1-4 and secondary tissue chambers 318B1-4 arranged in series. The pairs of primary/secondary tissue chambers 318A1-4 and 318B1-4 are all arranged in parallel between the fluid inlet 340 and the fluid outlet 372.

In another example shown in FIG. 2D, a microfluidic device 410 includes a first fluid inlet 440A feeding a first pair of primary/secondary tissue chambers 418A1, 418B-1 arranged in series, as well as a second part of primary/secondary tissue chambers 418A-2, 418B-2 arranged in series. The first and second pairs of primary/secondary tissue chambers are arranged in parallel with each other between the first fluid inlet 440A and fluid outlet 472. The microfluidic device 410 further includes a second fluid inlet 440B feeding a third pair of primary/secondary tissue chambers 418A-3, 418B-3 arranged in series, as well as a fourth pair of primary/secondary tissue chambers 418A-4, 418B-4 arranged in series. The third and fourth pairs of primary/secondary tissue chambers are arranged in parallel with each other between the second fluid inlet 440B and the fluid outlet 472. The first, second, third and fourth pairs of tissue chambers are arranged in parallel between the first and the second fluid inlets 440A, B and the fluid outlet 472.

FIG. 2E is a schematic overhead view of an example of a microfluidic device 111A including a plurality of tissue chambers 118 and 162 in a series arrangement. Microfluidic device 111A may be similar to microfluidic device 110 of FIG. 2A. However, microfluidic device 111A may include bent portions 173A and 173B of microfluidic channels 132 and 134, respectively. Bent portions 173A and 173B may form similar angles or different angles. Although the bend of bent portions 173A and 173 are shown to be a sharp bend, a curve of any radius may be provided in other examples.

The diameter A of primary tissue chamber 118 or secondary tissue chamber 162 may be selected based on the desired sizes of tissue to be placed within the respective tissue chamber. In one example, the diameter A is approximately 2 mm. The diameter A may be selected in a range from approximately 100 micros to approximately 4 mm in some examples. The width of microfluidic channels 132 or 134 may be similar or different. In some examples, the width B of microfluidic channels 132 or 134 may be approximately 375 microns. The width B may be selected in a range from approximately 100 micros to approximately 1 mm in some examples. In some examples, the width B may be set within a predetermine ratio, or ratio range, of height to width. Example ratios of height to width may be between 1:1 and 5:1 height to width in some examples.

FIG. 2F is a schematic overhead view of an example of a microfluidic device 111B including a plurality of tissue chambers 119 and 163 in a series arrangement.

Microfluidic device 111B may be similar to microfluidic device 110 of FIG. 2A and microfluidic device 111A of FIG. 2E. However, microfluidic device 111B may include a non-circular tissue chamber shape. Tissue chambers 119 and 163 may be similar to primary tissue chamber 118 and secondary tissue chamber 162, respectively, but tissue chambers 119 and 163 may have straight walls which result in a more narrow and non-circular tissue chamber shape. The length D and width C of tissue chambers 119 or 163 may be selected based on the desired sizes of tissue to be placed within the respective tissue chamber. In one example, the length D is approximately 2 mm. The length D may be selected in a range from approximately 100 micros to approximately 4 mm in some examples. In one example, the width C is approximately 1.3 mm. The width C may be selected in a range from approximately 100 micros to approximately 4 mm in some examples, with the width C being less than the length D in some examples, or vice versa in other examples.

FIG. 2G is a schematic overhead view of an example of microfluidic device 111C including a plurality of tissue chambers 120 and 164 in a series arrangement. Microfluidic device 111C may be similar to microfluidic devices 110 of FIGS. 2A and 111A of FIG. 2E. However, microfluidic device 111C includes a non-circular tissue chamber shape that enables a greater number of posts 115 for tissue chamber 120 and posts 161 for tissue chamber 164 which creates additional channels between the posts between the tissue chambers and microfluidic channels. Tissue chambers 120 and 164 may be similar to primary tissue chamber 118 and secondary tissue chamber 162, respectively, but tissue chambers 120 and 164 may have straight walls which result in a non-circular tissue chamber shape and more posts as discussed above. The length E and width C of tissue chambers 120 or 164 may be selected based on the desired sizes of tissue to be placed within the respective tissue chamber. In one example, the length E is approximately 2 mm. The length E may be selected in a range from approximately 100 micros to approximately 4 mm in some examples. In one example, the width F is approximately 1.8 mm. The width F may be selected in a range from approximately 100 micros to approximately 4 mm in some examples, with the width F being less than the length E in some examples.

FIG. 2H is a schematic overhead view of an example of microfluidic device 111D including a plurality of rectangular tissue chambers 121 and 165 in a series arrangement. Microfluidic device 111D may be similar to microfluidic device 110 of FIG. 2A and microfluidic device 111A of FIG. 2E. However, microfluidic device 111D may include a rectangular tissue chamber shape. Tissue chambers 121 and 165 may be similar to primary tissue chamber 118 and secondary tissue chamber 162, respectively, but tissue chambers 121 and 165 may have straight walls and posts arranged in a straight line which result in a rectangular, non-circular, tissue chamber shape. The aspect ratio of the length to width may be within the range of 1:1 to 1:4, or other ratio as desired for the sample tissue or other factors.

FIG. 2I is a schematic overhead view of an example of microfluidic device 111E including a single tissue chamber 122. Microfluidic device 111E may be similar to microfluidic device 110 of FIG. 2A and microfluidic device 111A of FIG. 2E. However, microfluidic device 111E may include only one, or a singular, tissue chamber 122.

Tissue chamber 122 is shown having a circular shape, but other non-circular shapes, such as a rectangular shape, in other examples of devices with only one tissue chamber.

FIG. 2J is a schematic overhead view of an example of microfluidic device 111F including a single tissue chamber 123 and a plurality of vascularization channels in parallel. Single tissue chamber 123 may be similar to tissue chamber 122 of FIG. 2I, but microfluidic device 111F may include two or more tissue chambers in parallel or series in other examples. After passing tissue chamber 123, microfluidic channel 174 passes along side and enables fluid communication with adjacent microfluidic channels 176A and 176B. Fluid inlet 144A leads to microfluidic channel 176A which ends at outlet 184A. Similarly, fluid inlet 144B leads to microfluidic channel 176B which ends at outlet 184B. Both of microfluidic channels 176A and 176B are in fluid communication with microfluidic channel 174 via separation elements 180A and 180B, respectively. Separation elements 180A and 180B may include a series of posts, for example, that create a plurality of channels through which fluid and/or cells may migrate between microfluidic channels 174 and 176A and between microfluidic channels 174 and 176B. In some example, fibrin and other cell solutions may be added to inlets 144A and 144B and create a gel within microfluidic channels 176A and 176B, respectively. The widths G, H, and I of microfluidic channels 176A, 174, and 176B may be the same and selected from a range of approximately 100 microns to approximately 4 mm. In one example, widths G, H, and I may be approximately 1.3 mm. Widths G, H, and I may be the same or different in other examples.

Channels 138A and 138B are disposed along microfluidic channels 176A and 176B, respectively, are fed via respective inlets 142A and 142B and end at respective outlets 182A and 182B. Serpentine portions 136A and 136B are disposed upstream of channels 138A and 138B, respectively. Both of microfluidic channels 176A and 176B are in fluid communication with microfluidic channels 138A and 138B via separation elements 178A and 178B, respectively. Separation elements 178A and 178B may include a series of posts, for example, that create a plurality of channels through which fluid and/or cells may migrate between microfluidic channel 176A and channel 138A and between microfluidic channel 176B and channel 138B. In some examples, channels 138A and 138B may be configured to accept fibrin and endothelial cells that were added to the respective inlet and flow through the respective channel where a gel is created. In this manner, the resulting fibrin and cell solution may provide the creation of a blood vessel network within channels 138A and 138B. The width J may be approximately 400 microns in some examples, or selected from a range from approximately 100 micros to approximately 2 mm in some examples. The width J of channels 138A and 138B may be similar or different.

FIG. 2K is a schematic overhead view of an example of microfluidic device 480A including a plurality of tissue chambers 484 in a series arrangement. Microfluidic device 480A may be similar to microfluidic device 110 of FIG. 2A and microfluidic device 111A of FIG. 2E. However, microfluidic device 480A only includes a single microfluidic channel 486A which may be similar to microfluidic channel 132 of FIG. 2A. In microfluidic device 480A, fluid inlet 481 is located upstream of serpentine portion 482 of microfluidic channel 486A. A series of three tissue chambers 484 (although fewer or greater tissue chambers may be used in other examples) are in communication with microfluid channel 486A via a plurality of channels 485 formed between posts 483. Media outlet 482 is then located downstream of the series of tissue chambers 484

Tissue chambers 484 may be similar to primary tissue chamber 118 and secondary tissue chamber 162, respectively, of FIG. 2A, but tissue chambers 484 may have two opposing straight walls result in a non-circular tissue chamber shape. The length L and width K of tissue chambers 484 may be selected based on the desired sizes of tissue to be placed within the respective tissue chamber. In one example, the length L is approximately 2 mm. The length L may be selected in a range from approximately 100 micros to approximately 4 mm in some examples. In one example, the width K is approximately 2 mm. The width K may be selected in a range from approximately 100 micros to approximately 4 mm in some examples. The width K and length L may be the same or different, e.g., the width K may be greater than or less than length L in other examples. The ratios of length to width may be selected from a range of 1:1 to 1:4 in some examples, or other ratios in other examples.

FIG. 2L is a schematic overhead view of an example of microfluidic device 480B including a plurality of tissue chambers 484. Microfluidic device 480B may be similar to microfluidic device 480A of FIG. 2K, but microfluidic device 480B includes additional tissue chambers 484. Microfluidic channel 486B is similar to microfluidic channel 486A, but microfluidic channel 486B is longer to accommodate six tissue chambers 484 instead of three. In addition, microfluidic channel 486B includes curved portion 487 between two of tissue chambers 484. Curved portion 487 is shown with a 180 degree curve, but other greater or smaller curvatures, and different radius of curvature, may be used in other examples. In other examples, microfluidic channel 486B may be straight or include two or more curved portions. This curvature in a microfluidic channel may be used for any of the microfluidic devices described herein. Although tissue chambers 484 are shown disposed along straight sections of microfluidic channel 486B, one or more tissue chambers 484 may be in fluid communication with microfluidic channel 486B along the concave or convex surface of a curved microfluid channel 486B, such as curved portion 487, in other examples. For any microfluidic devices described herein, tissue chambers may be located on straight or curved portions of a microfluidic channel.

FIG. 2M is a schematic overhead view of an example of microfluidic device 480C including a plurality of tissue chambers 484. Microfluidic device 480C may be similar to microfluidic device 480B of FIG. 2L, but microfluidic device 480C includes additional tissue chambers 484. Microfluidic channel 486C is similar to microfluidic channel 486B, but microfluidic channel 486C is longer to accommodate nine tissue chambers 484 instead of six. In addition, microfluidic channel 486C includes curved portion 487 and another curved portion 488 between respective pairs of tissue chambers 484. Curved portion 487 is shown with a 180 degree curve, and curved portion 488 is shown with a 180 degree curve in the opposite direction to create a serpentine shape for microfluidic channel 486C. However, in other examples, microfluidic channel 486C may include any number of curves and in any direction to create a desired flow profile for the tissue chambers and/or create a footprint desired to fit on the substrate supporting microfluidic device 480C.

Multiple microfluid devices described herein, such as microfluidic device 110, 111D, and 480A in some examples, include multiple tissue chambers. These tissue chambers are illustrated as being the same for each device. However, in other examples, a single microfluidic device may include multiple tissue chambers where at least one of the tissue chambers is different than another tissue chamber. For example, one tissue chamber may be circular while another tissue chamber may be rectangular. In other examples, the tissue chambers may have different dimensions, cross sectional areas, shapes, or even different post or channel sizes that allow different diffusion rates between the respective tissue chambers and microfluid channels.

FIG. 2N is a schematic overhead view of an example of microfluidic device 490 including a plurality of tissue chambers 118 and 162 and respective support channels 492 and 495. Microfluidic device 490 may be similar to microfluidic device 110 of FIG. 2A. However, microfluidic device 490 may include support channel 492 in fluid communication with tissue chamber 118 and support channel 495 in fluid communication with tissue chamber 162.

As shown in the example of FIG. 2N, inlet 491A enables fluid and/or substances to be added to support channel 492 which flows adjacent to tissue chamber 118 and then towards outlet 491B. The walls between support channel 492 and tissue chamber 118 define flow channel 493 which enables fluid and/or substances to migrate between support channel 492 and tissue chamber 118. For example, support channel 492 may be utilized to remove fluid from tissue chamber 118 and/or deliver nutrients, drugs, etc. to a tissue sample within tissue chamber 118. Similarly, inlet 494A enables fluid and/or substances to be added to support channel 495 which flows adjacent to tissue chamber 162 and then towards outlet 494B. The walls between support channel 495 and tissue chamber 162 define flow channel 496 which enables fluid and/or substances to migrate between support channel 495 and tissue chamber 162. For example, support channel 495 may be utilized to remove fluid from tissue chamber 162 and/or deliver nutrients, drugs, etc. to a tissue sample within tissue chamber 162. The width or cross-sectional area of support channels 492 and 495 may be similar or different. The width or cross-sectional area of support channels 492 and 495 may be similar or different from the width or cross-sectional area of microfluidic channels 132 and 134.

One or more flow channels (e.g., flow channel 493 or 496) may be defined between each tissue chamber and the respective support channel. In some examples, the width or cross-sectional area of the flow channels may be defined to act as a filter such that some substances cannot pass between the tissue chamber and support channel, such as reducing or precenting cell migration from the tissue chamber to the adjacent support channel. In some examples, the flow channel may have a smaller cross-sectional dimension than the passages between the posts separating microfluidic channels and the tissue chambers. As shown in the example of FIG. 2N, feeding channels similar to feeding channels 492 and 495 may be included for any of the microfluidic devices and tissue chambers described herein.

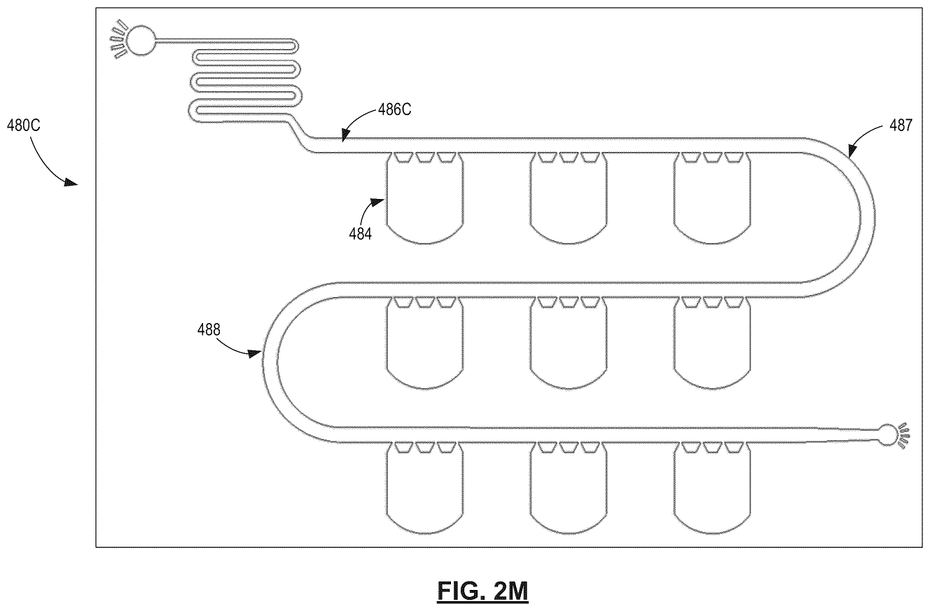

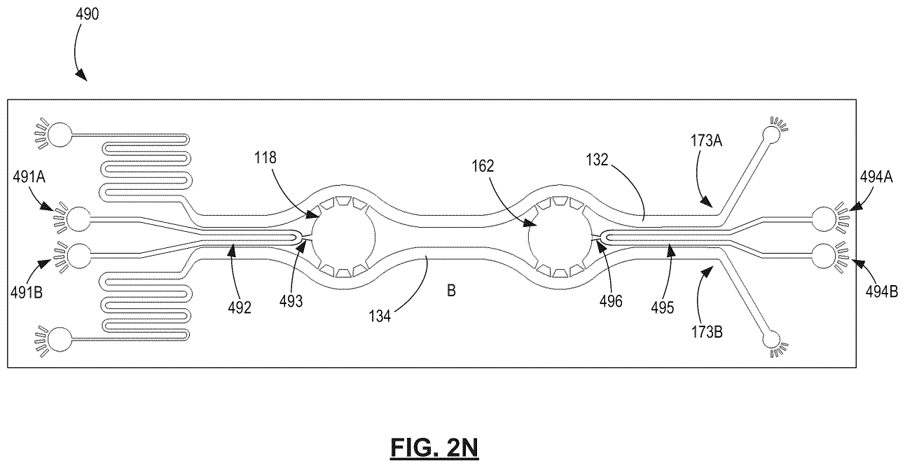

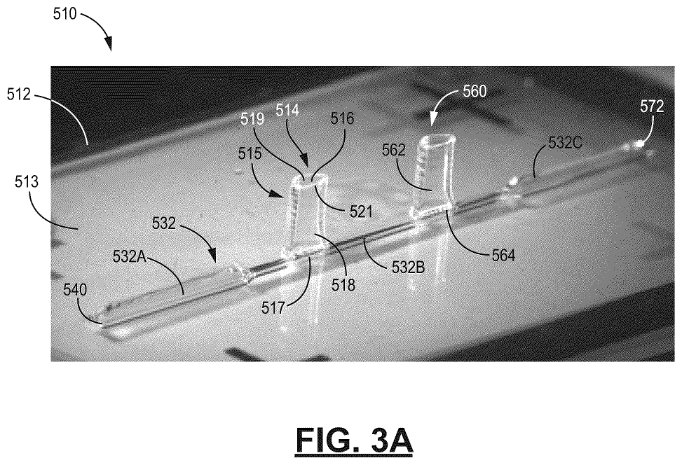

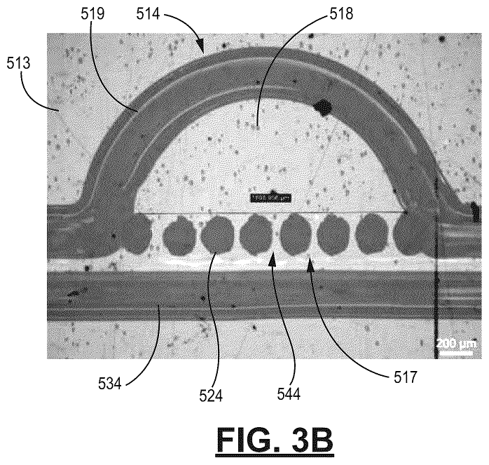

Referring now to the example of FIG. 3A, a 3D printed microfluidic device 510 includes a substrate 512 with a major surface 513. In the example of FIG. 3A, the substrate 512 is a glass slide, although as noted above the substrate may also include metals, polymeric films, plastics and the like. The device 510 includes a fluid inlet 540 upstream of a first portion 532A of a microfluidic channel 534. The microfluidic channel 532A is fluidly connected to a first body 514 that extends upward from the surface 513 of the substrate 512. The first body 514 includes a wall 515 with an arcuate portion 519 and a linear portion 521. The first body includes an open-facing port 516 and an open outlet 517. The open-facing port 516 is configured to allow insertion of a tumor spheroid or tissue sample with a diameter of at least 500 microns.

Referring also to FIG. 3B, the first body 314 includes a primary tissue chamber 518 within the enclosed wall 515 and between the open-facing port 516 and the open outlet 517. In the example of FIGS. 3A-3B, the primary tissue chamber 518 has a diameter at its widest point of about 1700 microns. The primary tissue chamber 518 includes an arrangement of posts 524 extending upward from the surface 513 of the substrate 512. The posts 524 are generally cylindrical and have a substantially circular cross-sectional shape when viewed from above through the open-facing port 516. In the example of FIGS. 3A-3B, the primary tissue chamber 518 includes eight posts 524, although as discussed above the number and size of the posts may vary widely depending on the extracellular matrix material to be retained in the primary tissue chamber 518, the insertion rate of the extracellular matrix material into the tissue chamber 518, and the like. In the example of FIGS. 3A-3B, the posts 524 have a diameter of about 200 microns and are spaced about 25 microns apart. The posts 524 are separated by channels or gaps 544. The posts 534 separate the primary tissue chamber 518 from the microfluidic channel 532A, which extends laterally with respect to the body 514.

A second portion 532B of the microfluidic channel 534 downstream of the first body 514 is fluidically connected to a second body 560 arranged in series with the first body 514. In the example of FIGS. 3A-3B, the second body 560 is substantially similar in shape and size to the first body 514, but the second body 560 may have any desired shape and size. The second body 560 includes a secondary tissue chamber 562, as well as an arrangement of posts 564 between the tissue chamber 562 and the microfluidic channel 532.

A third portion of the microfluidic channel 532C is located downstream of the second body 560, and is fluidically connected to a media outlet 572.

As noted above, the device 510 may be formed using a 3D printing or additive manufacturing process. One example of a suitable 3D printing process is described in U.S. patent application Ser. No. 16/951,794, which is incorporated by reference herein in its entirety.

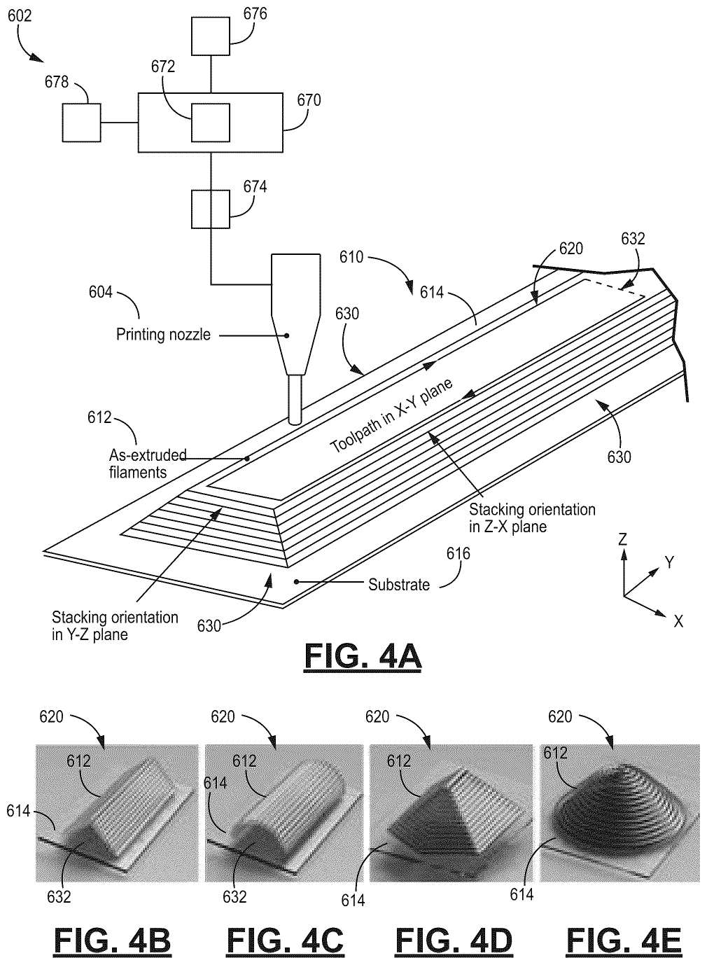

Referring now to FIG. 4A, a three-dimensional (3D) printing system 602 for extruding a 3D printed structure 610 is formed by extruding from a printing nozzle 604 an arrangement of overlying elongate polymeric filaments 612 on a surface 614 of a substrate 616.

In various examples shown schematically in FIG. 4A, the printing nozzle 604 in the system 602 may be interfaced with a controller 670 having at least one processor 672. The controller 670 may be configured to control one or more parameters of the printing nozzle 604 to determine one or more physical or chemical properties of the polymeric filaments 612 extruded from the nozzle 604. In some examples, which are not intended to be limiting, the controller 670 may be configured to mathematically reconstruct the surface geometry of a target surface, design routing and geometry of microfluidic channels based on the mathematical reconstruction of the surface geometry, to adjust one or more of the feed rate of a polymeric material to the printing nozzle 604, to adjust an angle of the printing nozzle 604 with respect to a plane of the surface 614, to form the toolpath of the printing nozzle 604 to create a desired pattern of the extruded polymeric material in the plane of the surface 614, or to move the printing nozzle 604 in one or more planes normal to the plane of the surface 614 to stack the filaments 612 on one another to form wall-like structures and enclosed passages.

In some examples, the controller 670 may be configured to process detected signals from one or more sensor systems 674 in or on the system 602. The processor 672 may be integrated with the sensor systems 674, may be integrated with the controller 670, or may be a remote processor functionally connected to the controller 670.

In some examples, the processor 672 may be coupled to a memory device 676, which may be part of the controller 670 or remote thereto. The memory device 676 may be used by the processor 672 to, for example, store fiducial information or initialization information corresponding to, for example, surface geometries, microfluidic channel designs, measurements or stored signals from the sensor system 674 of parameters of the system 602, the filaments 612, and the structures 610 formed therefrom. In some examples, the memory device 676 may store determined values, such as information corresponding to detected viscosity measurements for the extruded polymeric material, extrusion rates, toolpath patterns, and the like, for later retrieval. In some examples, the controller 670 and the processor 672 are coupled to a user interface 678, which may include a display, user input, and output (not shown in FIG. 4A).

The controller 670 can be configured to control any selected number of functions of the extrusion apparatus 602 including, but not limited to, toolpath patterns for the printing nozzle 604 considering channel width, filament diameter, wall incline angle, and overlapping of adjacent overlying filaments, extrusion rates for the polymeric materials extruded from the printing nozzle 604, and the like, in response to signals from the processor 672 input manually into the controller 670, or stored in the memory device 676. For example, in some examples, the controller may be used to mathematically reconstruct the target surface geometry and design the routing and geometry of microfluidic channels to incorporate pre-deposited elements, and to generate continuous and conformal printing toolpaths considering channel width, filament diameter, wall incline angle and overlapping of adjacent filaments. The controller 611s, encapsulate valves and pumps, and cut openings in the walls as need to insert connection tubes, and apply sealants to provide airtight or liquid-tight connections.

In FIG. 4A, the surface 614 of the substrate 616 occupies the x-y plane, and the filaments 612 are applied in a toolpath pattern 620 on the surface 614. In the example of FIG. 4A, the toolpath 620 when viewed from a perspective above the x-y plane of the surface 614 includes an arrangement of substantially linear filaments 612, but the toolpath pattern 620 may include circular patterns, arcuate patterns, trapezoidal patterns, and combinations thereof. For example, in FIGS. 4B-4C the filaments 612 are arranged in substantially linear toolpath patterns when viewed above the x-y plane, while in FIG. 4D the filaments 612 are arranged in a trapezoidal toolpath pattern 620, and FIG. 4E the filaments 612 are arranged in a substantially circular toolpath pattern 620.

Referring again to FIG. 4A, to form the 3D printed structure 610, the filaments 612 are stacked on each other along their longest dimensions (lengths) such that adjacent filaments overlie and adhere to one another to form a wall-like structure 630. As shown in FIG. 4A, the filaments 612 may be stacked in adjacent parallel planes normal to the x-y plane (the z-x plane and the y-z plane) to form the enclosed structure 410, which includes an internal passage 632.

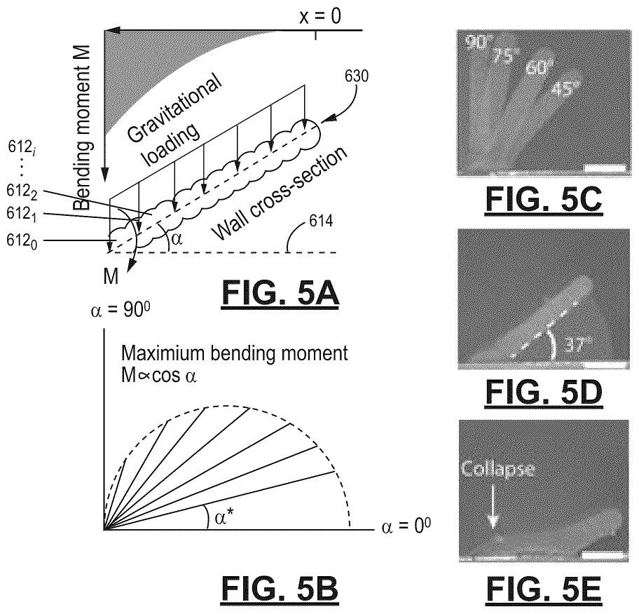

As shown in FIG. 5A, a cross-section of an example of a wall structure 630 as viewed in a plane normal to the plane of the surface 614 of the substrate 616 (for example, the y-z plane normal to the x-y plane in FIG. 4A) includes a first elongate filament 612₀ on the surface 614 of the substrate 616. To form the wall 630, a series of filaments 612₁-612ₙ are formed on the first elongate filament 612₀. The nozzle 604 moves along an angle in the plane normal to the x-y plane and extrudes each filament 612₁-612ₙ formed in a plane substantially parallel to the x-y plane of the first filament 612₀. The filaments 612₀-612ₙ adhere to each other along their lengths to form the wall structure 630. While the example filaments 612₀-612ₙ in FIG. 5A are shown with a substantially circular cross-sectional shape, depending on the polymeric materials selected to form the filaments 612 and a cross-sectional shape of the extrusion nozzle 604 (FIG. 4A), many different cross-sectional shapes are possible, including regular shapes such as squares, trapezoids, ovals and the like, as well as irregular shapes. In various examples, which are not intended to be limiting, the filaments 612 have a cross-sectional diameter of about 100 nm to about 500 m, or about 100 nm to about 200 m, or about 100 nm to about 100 μm.

The composition utilized to make the filaments 612, which can also be referred to as an ink, can vary widely depending on the intended use of the printed structure 610. Suitable ink materials should include at least one polymeric material with suitable yield strength, elastomeric properties, and good adhesion to surfaces. In various examples, which are not intended to be limiting, the polymeric materials can include silicones, (meth)acrylates (wherein (meth)acrylate includes acrylates and methacrylates) such as polymethylmethacrylate (PMMA), polystyrene, poly(ethylene glycol) diacrylate (PEGDA), polymeric materials and gels available from Lubrizol Life Science, Bethlehem, PA under the trade designation CARBOPOL, hydrogels, and biodegradable polymers such as polylacticcoglycolic acid (PLGA), biocompatible polymers available from Eden Microfluidics, Paris, FR, under the trade designation FLEXDYM, and thiolenes.

In some examples, the ink includes a silicone compound, which may be hardenable at room temperature, with heat, or with radiation such as, for example, UV light. In some examples, the silicone utilized to make the filaments 612 is an acetoxy silicone that is room temperature vulcanizing (RTV) when exposed to moisture in the air.

In one example, which is not intended to be limiting, a suitable silicone compound for the ink is a one-part acetoxy silicone available under the trade designation LOCTITE SI 595 CL from Henkel, AG, Minneapolis, MN. This one-part silicone does not require prior mixing or other preparation and cures in ambient environment without requiring UV irradiation or thermal heating. In addition, cured RTV silicone structures made from acetoxy silicones demonstrate high elongation before breaking and good adhesion to different surfaces. In some examples, which are not intended to be limiting, the acetoxy silicone has a Young's modulus of about 10 kPa to 10 MPa, or 150 kPa to about 250 kPa, or about 175 kPa to about 200 kPa, or about 190 kPa.

In some examples, the printed structure 610 of FIG. 4A can include optional sacrificial materials, which can be used to temporarily support the structure 610 during, for example, printing and curing steps. For example, the sacrificial materials can be printed with a second ink different from the first polymeric ink, and can be used to temporarily fill hollow channels and chambers. The sacrificial materials are then removed from the printed structure during or after the curing or hardening process. Suitable sacrificial materials include, but are not limited to, pluronics (block copolymers including hydrophilic polyethyleneoxide (PEO) and hydrophobic polypropylene oxide (PPO) blocks), sugar networks, water soluble polymers, acrylonitrile butadiene styrene (ABS), paraffin-based inks such as, for example, Prussian blue paste, petroleum jelly, microcrystalline wax, carbohydrate gels, hydrogels, liquid metals, and mixtures and combinations thereof.

As shown in FIGS. 5A-5B, when viewed in a plane normal to the plane of the surface 614 to which filament 612₀ is applied (for example, the y-z plane normal to the x-y plane in FIG. 4A), the wall 630 forms a wall angle α with respect to the plane of the surface 614. For as-printed walls 630 in the sub-millimeter regime, the yield strength of the as-printed silicone-containing ink is sufficient to balance the bending moment induced by gravity within a predetermined angular range. As shown in the examples in FIGS. 5C-5E, in various examples the wall angle α can vary widely from about 350 to about 90°, or about 370 to about 90°, or about 450 to about 90°, or about 370 to about 75°, or about 450 to about 60°.

As shown schematically in FIGS. 5A-5B, the wall 630 formed from the silicone-containing ink composition has a maximum bending moment M proportional to cos(a), and if the wall has a length of about 700 m and wall angle α greater than about 35°, the wall 630 will effectively resist gravitational loading exerted in a direction normal to the surface 614 and form a self-supporting wall structure. However, if the wall angle α drops below about 35°, depending on factors such as, for example, the polymeric material used to form the filaments 612, and the cross-sectional dimensions of the filaments 612, in some cases the wall 630 cannot support its own weight, and the gravitational loading causes the wall to collapse (FIG. 5E).

While not wishing to be bound by any theory, presently available evidence indicates that an uncured RTV (room temperature vulcanizing) silicone such as LOCTITE SI 595 CL exhibits the mechanical behavior of a yield-stress fluid, with storage modulus greater than loss modulus at low frequency. This renders a yield stress that must be overcome to initiate flow under the gravitational loading. Because the RTV silicone starts curing instantaneously after dispensing, as evidenced by increasing storage modulus over time, the predicted yield strength is slightly higher than the measured value. RTV silicone also exhibits shear thinning behavior in the uncured state. Briefly, in some example examples, which are not intended to be limiting, RTV silicones such as LOCTITE SI 595 CL have an apparent viscosity of about 104 Pa·s at a shear rate of 0.01 s⁻¹, and the viscosity decreases to about 10-3 Pa·s as the shear rate increases to 1000 s⁻¹. This variation in viscosity leads to a relatively low dispensing pressure through the nozzle 604 of about 175 psi with 100 μm printing nozzles, and a stronger resistance to creep for the as-printed structures.

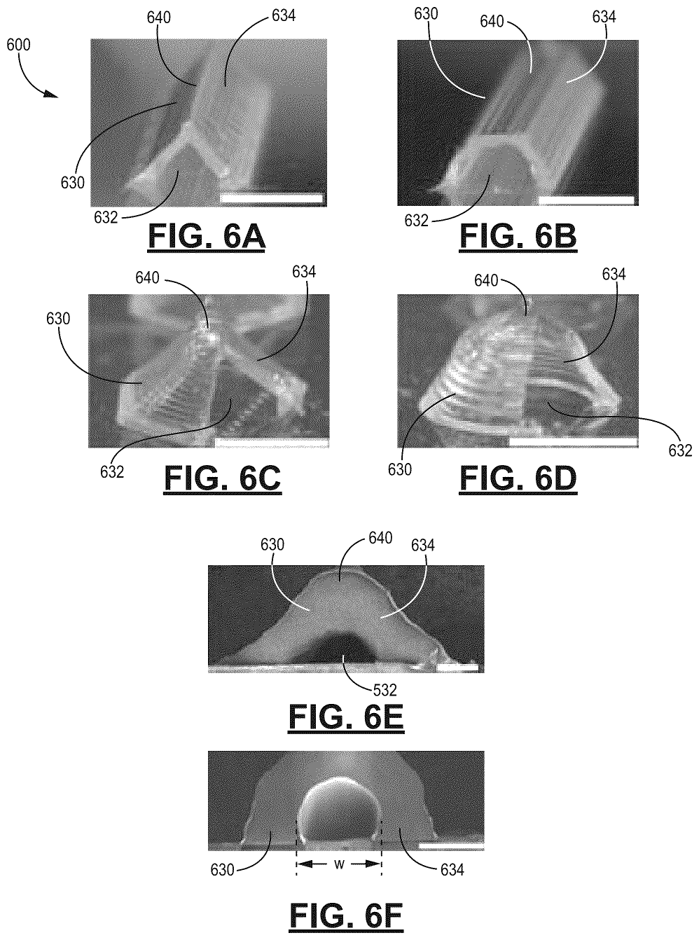

Aa shown in FIGS. 6A-6D, enclosed structures 600 could be formed from opposed silicone walls 630, 634 that are configured to meet in a roof-like apex region 640. As shown in FIG. 6A, in some examples the apex region 640 is a narrow seam formed from a single filament or a small number of filaments, and in other examples shown in FIG. 6B the apex region 640 is a relatively wide, flat, planar portion of the structure 600 that itself is formed from multiple filaments. In other examples, the apex region 640 can be point or a narrowing frustoconical region atop the opposed walls 630, 634 (FIGS. 6C-6D). The enclosed structures 600 each include a continuous fluid passage 632, which can have a wide variety of cross-sectional shapes including triangular (FIG. 6A), trapezoidal (FIG. 6B), pyramidal (FIG. 6C), conical (FIG. 6D), square, and the like.

The geometries of the enclosed fluid passages 632 are based on the toolpaths in the X-Y plane and the vertical stacking angle of the filaments used to form the opposed walls 630, 634 (FIGS. 6A-6C). The dimensions of the fluid passages 632 can be selected by specifying the distance between the opposed sidewalls 630, 634 sidewalls and the incline angle α. The printing toolpath 620 (FIG. 6A) is determined based on, for example, the thicknesses of the extruded filaments 612, calibrated with the size of the printing nozzle 604 and the extrusion pressure of the polymeric material. In various examples, which are not intended to be limiting, the fluid passages 632 have an inner cross-sectional width w of about 100 μm to about 500 μm (FIGS. 6E-6F).

In some cases, each of the walls 630, 634 may include the same number of filaments, but in other examples one or both walls may be designed to include an extra filament or group of filaments referred to herein as a spacer filament. The number, location, and size of the spacer filaments may be selected to modify the shape of the wall, the shape of the internal passage 632 between the opposed walls, or both. The spacer filaments can be formed from the same polymeric materials as the other filaments forming the walls 630, 634, or may be formed from a different polymeric material.

In some examples, overlying adjacent filaments used to form the walls 630, 634 adhere to each other along their lengths to form a fluid-impermeable structure. If the opposed walls 630, 634 and the apex region 640 are fluid-impermeable, the enclosed fluid passages 632 within the structures 600 form self-supporting microfluidic devices. In various examples, the microfluidic structures 600 made from silicone-containing inks have a burst pressure of about 20 kPa to about 80 kPa, or about 20 kPa to about 50 kPa, or greater than about 40 kPa, at wall thicknesses of about 100 μm to about 400 μm. For example, at a wall thickness of about 200 μm, burst pressures fall in the range of about 30 kPa to about 50 kPa. For comparison, the backpressure applied to actuate aqueous flows in most microfluidic applications rarely exceeds about 10 kPa. While not wishing to be bound by any theory, computational fluid dynamic (CFD) simulations indicate that the microfluidic structures 600 required back pressures below 1 kPa, and the devices can be used repeatedly with no observable leakage at backpressures at a pressure of 10 kPa to about 75 kPa.

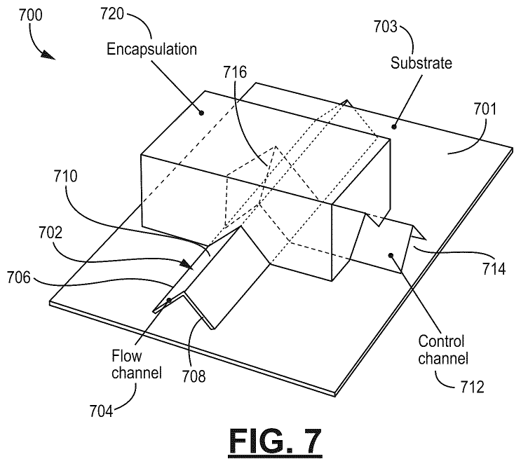

Referring now to FIG. 7, a 3D printed microfluidic valve 700 includes an elongate structure 702 extruded on a surface 701 of a substrate 703. The structure 702 is formed with a plurality of polymeric filaments stacked on each other along their lengths as described in FIG. 4A above, and the filaments enclose a flow channel 704 for transporting a liquid or a gaseous material. The structure 702 includes opposed liquid impermeable walls 706, 708 that merge in an apex region 710, each of the walls 706, 708 having a wall angle of greater than about 35°. In some examples, which are not intended to be limiting, the polymeric filaments used to make the structure 702 are formed from the RTV silicone materials described above.

A control channel 712 is conformally printed over a portion of the structure 702. The control channel 712, which was also 3D printed using stacked polymeric filaments as described above, also includes an enclosed fluid passage 714. In some examples, which are not intended to be limiting, the polymeric filaments used to make the flow channel 702 are formed from the RTV silicone materials described above. The control channel 712 further includes a valve section 716 that overlies the flow channel 704 at a crossing junction therewith. An enclosed hollow space is formed between the two channels 704, 712 to act as a valve when actuated by pressurized gas flowing through the fluid passage 714.

The valve section 716 is overlain and encapsulated by an encapsulant structure 720. The encapsulant structure can be formed from any suitable encapsulant material, and acrylate ester-based resins have been found to be particularly suitable. In some cases, the acrylate ester resins can be curable by radiation such as UV light. Given the presence of the overlying encapsulant structure 720, downward expansion of a pressurized gas in the control channel 712 acts on the flow channel 702 and closes the valve section 716. The highly elastic walls of the control channel 712 and the flow channel 702 thus form a flexible native membrane to open or close the valve section 716.

In some examples, the control channel 712 can be interfaced with external tubes (not shown in FIG. 7) and sealed directly with the encapsulation resin. Generally, a higher flow pressure in the flow channel 702 requires a correspondingly higher closing pressure in the control channel 712 to stop the flow. For example, in some examples, a 300 kPa controlling pressure in the control channel 712 closed the valve 716 completely while a hydraulic pressure up to 30 kPa was applied to the flow channel 702.

In another example, peristaltic microfluidic pumps can also be directly 3D printed using three controlling channels laid out in parallel and encapsulated as one unit. The microfluidic pump was operated by activating the control channels according a three-phase peristaltic code. Longer actuation times yielded a more complete shut-off of the control channels and therefore could be tuned to generate a higher pumping volume per cycle. In one example, a flow rate of 105 L/cycle was achieved with an actuation pressure of 100 kPa and an actuation time of 1.2 sec.

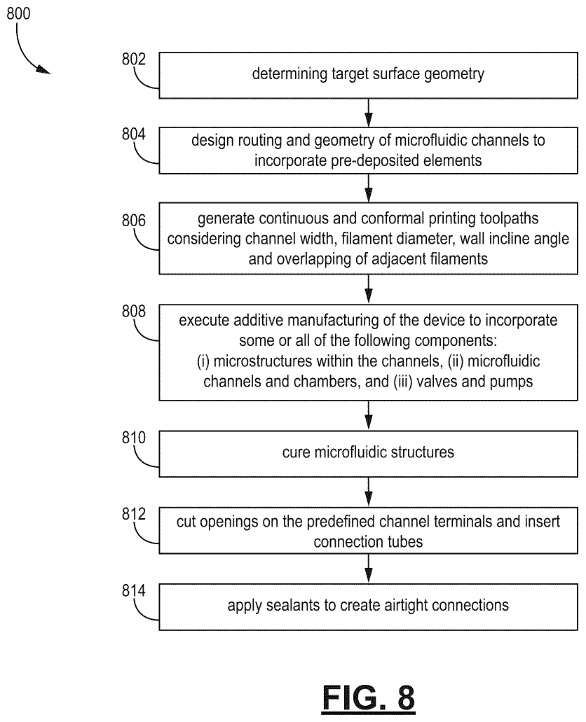

Referring now to FIG. 8, the present disclosure is further directed to a method 800 for 3D printing a microfluidic device on a substrate. In step 802, the method includes mathematically reconstructing the target surface geometry. In step 804, the method further includes designing and routing and geometry of microfluidic channels to incorporate pre-deposited elements. In step 806, the method further includes generating continuous and conformal printing toolpaths considering parameters such as, for example, channel width, filament diameter, wall incline angle and overlapping of adjacent filaments. In step 808, the method further includes executing 3D printing in the order of: (i) microstructures within the channels, (ii) microfluidic channels and chambers, and (iii) encapsulation materials for valves and pumps. In step 810, the method further includes curing the microfluidic structures. In optional step 812, the method further includes cutting openings on the predefined channel terminals and insertion of connection tubes. In optional step 814, the method includes applying sealants to create airtight connections. The excellent elasticity of the cured silicone channels enables a facile and tight connection to external tubing.

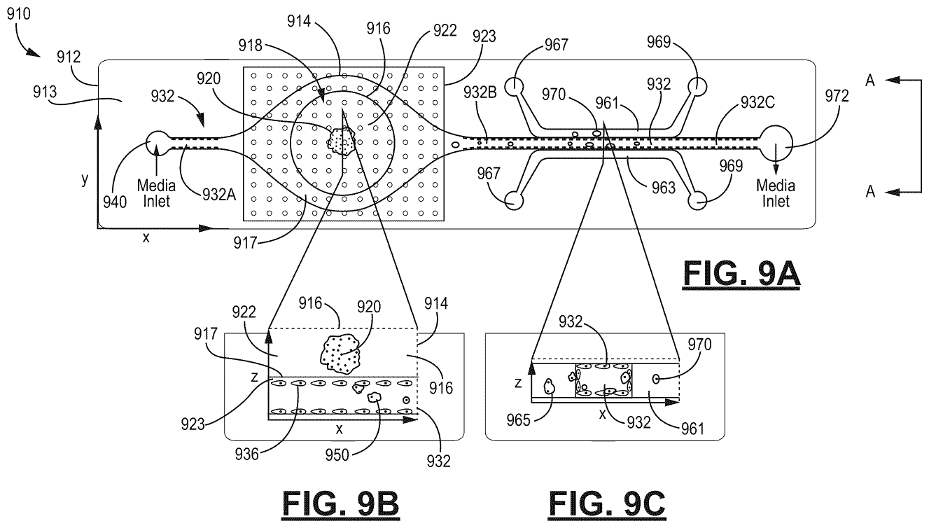

Referring now to FIGS. 9A-9B, in another example a microfluidic device 910 includes a substrate 912 with a major surface 913. As with the examples discussed above, the substrate 912 may be made from metals, polymers, plastic, or glass. Examples of suitable polymers for the substrate 912 include polydimethylsiloxane (PDMS), poly(methyl methacrylate) (PMMA), polystyrene (PS), SU-8, and cyclic olefin copolymer (COC). All or a portion of the substrate 912 and other portions of the device 910 are made of a biocompatible material, e.g., the material is compatible with, or not toxic or injurious to cells, tissue, and other living material.

The device 910 includes a body 914 extending away from the surface 913 of the substrate 912. In some examples, which are not intended to be limiting, the body can have a height of about 1 mm to about 5 mm above the surface 913. In various examples, the body 914 is made from any of the same biocompatible materials as the substrate 912, and may be formed by any suitable technique including, for example, injection molding, soft photolithography, 3D printing, or combinations thereof.

The body 914 includes an open-facing port 916 configured to allow introduction of large tissue samples into the body 914, and in some examples is sufficiently large to allow passage of tissue samples with a diameter of greater than about 500 microns, greater than about 1 mm, or greater than about 1.5 mm, or greater than about 2 mm. As noted above, the large open-facing port 916 makes possible the insertion of a large tumor or other tissue sample with a biopsy punch.

The body 914 further includes an open second end 917 opposite the open-facing port 916. The open second end 917 of the body 914 allows the contents of the body 914 to be in fluid communication with other microfluidic channels on the surface 913 of the substrate 912.

An interior region of the body 914 includes a tissue chamber 918 between the open-facing port 916 and the open second end 917. The tissue chamber 918 includes dimensions selected to contain a particular tumor or tissue sample for analysis. The dimensions of the tissue chamber 918 may vary widely, and in some examples the tissue chamber 918 has an inside diameter of about 50 microns to about 1 mm, or about 200 microns to about 2 mm. The types of tissues suitable for analysis in the tissue chamber 918 can also vary widely, and in some examples can include biologically relevant tumors such as, for example, a multicellular tumor spheroid (MCTS), a patient-derived xenograft (PDX) derived organoid, or a primary tumor biopsy. In addition to the larger tissue samples or tumors, the tissue chamber 918 can further be configured to contain other types of human or cancerous organoids, cell clusters, aggregates, individual cells, and mixtures and combinations thereof. The tissue chamber 918 further includes an extracellular matrix material 922.

The tumor microenvironment 910 further includes a media inlet 940, which is fluidly connected to a first portion 932A of a microfluidic channel 932. As described above with reference to FIG. 1, the microfluidic channel 932 has a diameter of about 50 microns to about 200 microns, and is some examples can be lined with a layer of endothelial cells 936.

A separation element including a membrane 923 underlies the open second end 917 of the body 914. Suitable membranes can vary widely, and in some examples a porous polymeric material with an average pore size of about 1 nanometer (nm) to about 500 microns, or about 50 nm to about 250 microns, or about 100 nm to about 200 microns, has been found to provide good performance. The membrane 923 retains the extracellular matrix material in the tissue chamber 918, while allowing passage of liquids, cells 950 and the like from the tissue chamber 918 to the microfluidic channel 932 that underlies the open second end 917 of the body 914.

A second portion 932B of the microfluidic channel 932 is downstream of the body 914, and enters a secondary body 960. As further shown in FIG. 9C, the secondary body 960 includes a first tissue chamber 961 on a first side of the microfluidic channel 932, and a second tissue chamber 965 on a second side of the microfluidic channel 932. The first and second tissue chambers 961, 965 are filled with an extracellular matrix material 963, and are in fluid communication with the microfluidic channel 932. In some examples, the first and second tissue chambers 961, 965 include optional fluid inlets 967 and fluid outlets 969.

As shown in FIG. 9C, cells or cell aggregates 970 migrate from the microfluidic channel 932 through the layer of endothelial cells 936 and enter the tissue chambers 961, 965. Once lodged in the tissue chambers 961, 965, the cells 970 can grow and develop, thus forming a tumor metastasis model for the tumor spheroid 920 in the primary tissue chamber 918.

The device of FIG. 9A further includes a third portion 932C of the microfluidic channel 932 fluidly connected to at least one media outlet 972, which may be connected to a reservoir or another arrangement of microfluidic channels (not shown in FIG. 9A).

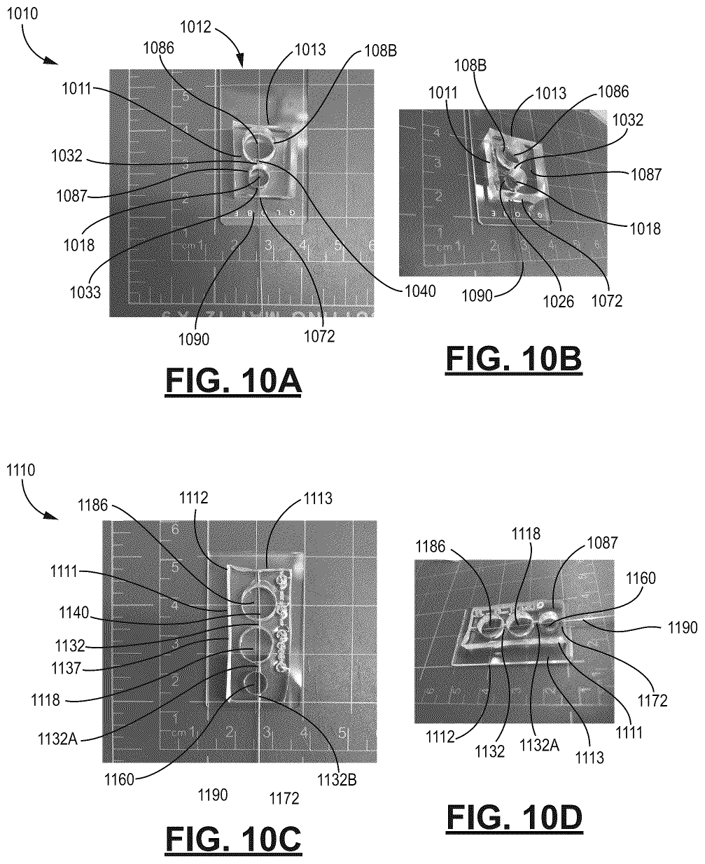

In yet another example shown in FIG. 10A-10B, a microfluidic device 1010 includes a transparent glass substrate 1012 formed from, for example, a microscope slide, although substrates of plastics, metals, and the like may also be used. A structure 1011 resides on a major surface 1013 of the substrate 1012. In the example of FIGS. 10A-10B, the structure 1011 is formed from a polymeric material by a method including, for example, molding, 3D printing, photolithography, machining, and the like. The structure 1011 includes a media reservoir 1086 formed therein and an exposed surface 1087. The media reservoir 1086 is bounded by a wall 1088 extending upward from the surface 1087. The media reservoir 1086 further includes a media inlet 1040.

The structure 1011 further includes a tissue chamber 1018 with a wall 1026 extending upward from the surface 1087. The tissue chamber 1018 includes an open top configured to allow introduction of a large tumor or tissue sample with a diameter of at least 500 microns via, for example, a biopsy punch. A microfluidic channel 1032 in the structure 1011 separates the media reservoir 1086 from the tissue chamber 1018.

A plug 1090 such as, for example, a metal pin, extends into structure 1011 and occupies the microfluidic channel 1032. When the plug 1090 is removed from the microfluidic channel 1032, media flows in the direction of the arrow A from the media reservoir 1086 into the microfluidic channel 1032 and into the tissue chamber 1018. After passing through the tissue chamber 1018, the media may proceed into a second microfluidic channel 1033 in the structure 1011 and into a media outlet 1072.

In another example shown in FIGS. 10C-10D, a microfluidic device 1110 includes a transparent glass substrate 1112 formed from, for example, a microscope slide, although substrates of plastics, metals, and the like may also be used. A structure 1111 resides on a major surface 1113 of the substrate 1112, and as described above can be formed from a polymeric material. The structure 1111 includes a media reservoir 1186 formed therein and an exposed surface 1187. The media reservoir 1186 further includes a media inlet 1140.

The structure 1111 further includes a primary tissue chamber 1118. The primary tissue chamber 1118 includes an open top for introduction of a large tumor or tissue sample with a diameter of at least 500 microns via, for example, a biopsy punch.

A microfluidic channel 1132 in the structure 1111 separates the media reservoir 1186 from the primary tissue chamber 1118. A second microfluidic channel 1132A in the structure 1111 extends from the primary tissue chamber 1118 to a secondary tissue chamber 1160.

A plug 1190 such as, for example, a metal pin, extends into structure 1111 and occupies the microfluidic channels 1132, 1132A. When the plug 1190 is removed from the microfluidic channels 1132, 1132A, media flows in the direction of the arrow A from the media reservoir 1186 into the microfluidic channel 1132, into the tissue chamber 1118, into the second microfluidic channel 1132A, and into the secondary tissue chamber 1160. After passing through the secondary tissue chamber 1160, the media may proceed into a third microfluidic channel 1132B in the structure 1111 and into a media outlet 1172.

In another aspect, the present disclosure is directed to methods of producing the microfluidic devices described above. In one example, the microfluidic devices are manufactured in two pieces. A top piece (e.g., of polymer, glass or plastic) includes etched microchannels formed by, for example, micromachining, photolithography, hot embossing, soft lithography, microinjection molding, or any other manufacturing technique. The etched channels make the “roof and” “walls” of the channel, and the “floor” of the channel includes a flat portion (e.g., of polymer, glass, or plastic) that is bonded to the top piece. The “floor” of the device be either made of the same or a similar material as used for the “roof” and “walls” of the device, or made of a different material. Bonding can be done in a variety of ways, such as epoxies or, in the case of PDMS and glass, by exposing both pieces to a plasma and then simply placing them in contact with each other.

Apertures are created in the top piece to allow fluid flow through the micro-channels. These holes can be placed at the ends of the micro-channels, or anywhere along their length. After the top and bottom pieces are bonded, it may or may not be necessary to flow a coating composition through the channels. In one example, a coating composition including poly-d-lysine may be washed through the channels to promote cellular adhesion and stabilize the gel. In some examples, the coating composition may be used to control the hydrophobicity of the channel walls, as some materials require additional surface treatments to allow a desired level of cell media flow.

The tissue chambers of the device can then be filled with an extracellular matrix material such as a gel, and surface tension will keep the gel from spilling out of the tissue chamber and entering the microchannels. In some examples, the gel is injected into the device via the gel filling port. Different pressure-time injection profiles for gel insertion may be used to improve the quality of the injected gel. For example, the gel may require an initial burst of injection pressure to get into the channel, then a sustained lower pressure to finish the gel filling. Alternatively, gel may be injected from both ends of the tissue chamber simultaneously.

In some examples, the extracellular matrix material is then allowed to harden (e.g., for one hour in a cell culture incubator), and the device is then ready for cell seeding.

Accordingly, in one aspect, the present disclosure is directed to a method of making a device including etching one or more microfluidic channels and one or more tissue chambers into a first portion of the optically transparent material. The method further includes creating one or more fluid inlets to allow flow through the one or more microfluidic channels and the one or more tissue chambers, which creates a roof and walls of the device; and bonding the first portion of the optically transparent material to a second portion of the optically transparent material that forms a floor of the device.

The method can further include introducing a coating composition through the microfluidic channels. Examples of suitable coating compositions include agents include, but are not limited to, glutaraldehyde, polyethyleneimine, poly-D-lysine, fibronectin, laminin, collagen, and mixtures and combinations thereof. The method can further optionally include treating the surfaces of the microfluidic channels to modify the hydrophobicity thereof. Suitable techniques for modifying hydrophobicity include chemical treatments or treatments with a plasma.

The method further includes introducing the extracellular matrix material into one or more of the tissue chambers, which can create a tumor microenvironment for the tissue sample or tumor resident therein.

The devices provided herein can be used for a variety of purposes. For example, cells (e.g., endothelial cells) can be introduced (e.g., injected) into the microfluidic channel by one of the media inlets. Fluid percolates through the microfluidic channels, sweeping endothelial cells with them onto the walls of the channels. The cells can be cultured for the desired number of days such as, for example about 1 day to about 7 days to form microfluidic channels lined with endothelial cells, mimicking a blood vessel wall.

In some examples, biological compounds such as drugs can be applied to the media inlet to attempt to retard the appearance or growth of sprouts toward the tumor spheroid or other tissue sample, or to enhance sprouting frequency and size. In another example, cancer cells (e.g., disassociated cells from a cancer patient's cancer biopsy) can be injected, along with a (one or more) potential anti-cancer spreading drugs or combinations of anti-cancer drugs, into the tumor microenvironment. If, after culturing the device, the cancer cells have not progressed toward the endothelial barrier or formed metastases, then the drug combination can potentially be considered a viable anti-cancer drug. If the cancer cells are disassociated cells from a cancer patient's cancer biopsy, then the drug or drug combination is not appropriate to stop the spread of (or treat) that patient's cancer.

As discussed above, in some examples several tissue chambers may be linked together in series or parallel, with separate or common fluid channels among them, which can allow multiple cancer drugs to be tested at once, or perhaps allow one tissue biopsy to be tested with many non-interfering anti-metastasis drugs as possible.

Cancer generally begins at a specific site in the body. When the cancer grows, the tumor must feed itself, and to do that, induces new blood vessels to grow within it, in a process called angiogenesis. When cancer spreads, in a process known as metastasis, individual or clusters of cancer cells will detach from the primary tumor and use the blood and lymphatic vessels to migrate to other parts of the body. To get into the blood stream, cancer cells must burrow through the blood vessel walls, particularly through endothelial cells, which line the blood vessel wall. This is known as intravasation. The cancer cells must burrow back through the blood vessel walls to get into the body's tissue after traveling, called extravasation. Once in a new tissue site, cancer cells must form lesions, which requires coordination of multiple stromal cell types. Angiogenesis, intravasation, extravasation, and colonization are all targets for anti-cancer drugs.

In the anti-angiogenesis drug discovery example, new anti-intravasation drugs may be placed in one of the outer channels, and standard cell culture media in the other outer channel as a control. The devices described herein allow for quantitative assessment of the drug's efficacy. In the anti-metastasis drug discovery example, the device can be treated as above, but with a cancer cells, either from a cell line or from a biopsy, to be seeded. The devices then allow easy imaging and, via downstream image processing, quantitative analysis of the number of cancer cells that have burrowed through the newly formed blood vessel sprouts, and how the process is retarded with potential anti-metastasis drugs.

In a personalized medicine example, biopsied cancer cells can be placed in the device and endothelial cells, from either the patient or a cell line, are allowed to form blood vessels. Several anti-intravasation drugs or anti-angiogenesis drugs can then be tested in these devices. Since it appears that different patients respond to different anti-metastasis drugs, these devices can be used to detect what drugs stop cancer cell dissemination and angiogenesis for that patient, and what drugs do not.

Accordingly, the microfluidic devices of the present disclosure and the tumor microenvironments formed therefrom can be used in methods such as, for example, identifying whether an agent is angiogenic or anti-angiogenic, determining whether a biological agent can be used to slow or prevent metastasis, determining whether blood vessels can grow toward a tumor spheroid in vitro, identifying whether an agent is chemoattractive agent or a chemorepulsive agent of neuronal cells, or a method of identifying whether a biological agent such as a drug can be used to treat cancer, and the like.

The devices of the present disclosure will now be further described in the following non-limiting examples.

### EXAMPLES

### Example 1

A microfluidic device similar to that shown in FIG. 1 was prepared from two component parts. The microfluidic device included two layers: a top polydimethylsiloxane (PDMS) layer with microfluidic channels and tissue chambers and a bottom glass slide. The master mold for the top PDMS layer was fabricated on a silicon wafer (thickness: 250 μm) with SU-8 negative photoresist patterned by ultraviolet (UV) photolithography. Uncured PDMS solution (Sylgard 184, Dow Corning) was casted on the mold and cured for 2 hours at 70° C. in an air-vented oven.

The cured PDMS was then peeled from the mold and inlets (diameter: 0.25 mm), outlets (diameter: 0.5 mm), and tissue wells (diameter: 2 mm) were punched on the cured PDMS using biopsy punches. The top PDMS and bottom glass layers were cleaned using an adhesive tape and treated with air plasma (1 min, 50 sccm, 40 mW) to ensure permanent bonding. Furthermore, the two-layer device was cured in an oven at 75° C. for more than 1 h to complete the bonding. The microfluidic device was then stored in a 60-mm dish and exposed to UV radiation for 1-2 hours before cell seeding experiments.

Cell suspensions of MDA-MB-231-GFP, Hs578T-GFP, BT-549GFP, MCF7-GFP and MCF-10A-mRuby were harvested in an ultra-low adhesion 96-well plate with U-shaped bottom well to prepare multicellular tumor spheroids for the experiments.

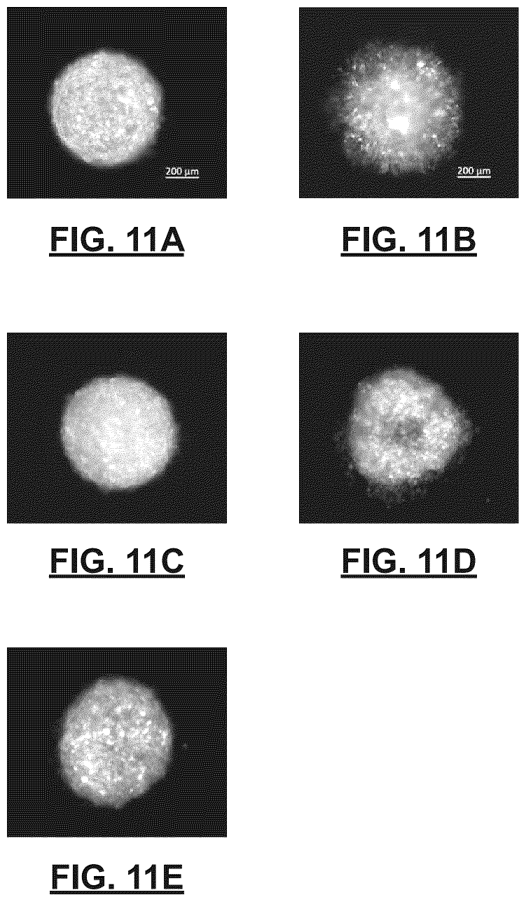

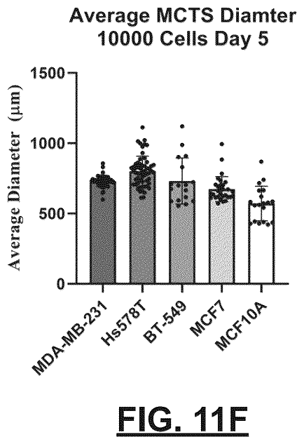

Monoculture spheroids composed of MDA-MB-231-GFP, Hs578T-GFP, BT-549GFP, MCF7-GFP, and MCF-10AmRuby cells were prepared with an initial seeding concentration of 10,000 cells/well in 100 μL of DMEM medium containing 2.5% Matrigel, which significantly caused the aggregate cells to form spheroids. Photographs of the spheroids are shown in FIGS. 11A-11E, and FIG. 11F is a plot of the average diameter of 10000 cells of the spheroids on day 5. At this dimension, oxygen and nutrients are limited in diffusion, which leads to the development of a necrotic core and proliferative outer shell, which are key features of tumors in vivo.

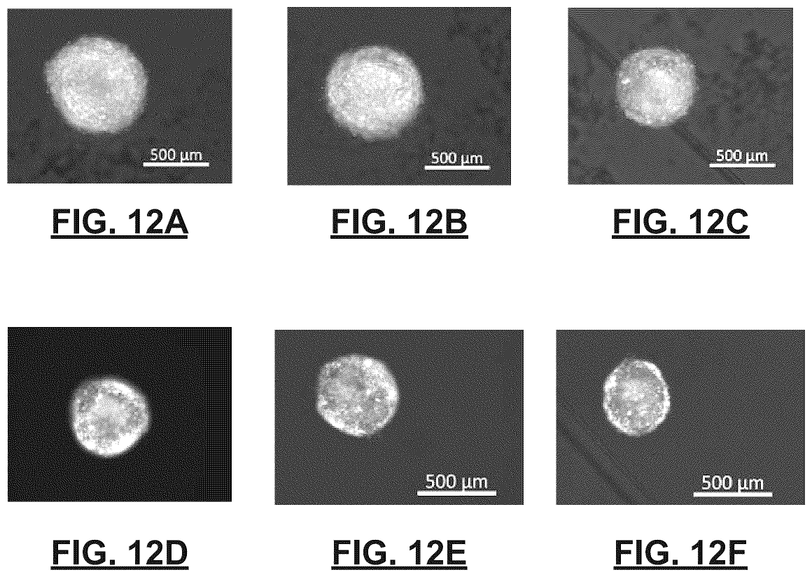

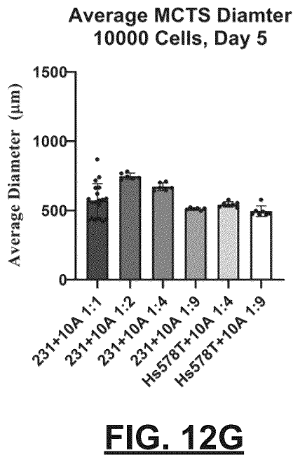

Furthermore, co-culture tumor spheroids including both cancer cells and normal cells were initiated by mixing MDA-MB-231-GFP or Hs578T-GFP cells with MCF-10A-mRuby cells (at ratios of 1:1, 1:2, 1:4, and 1:9; total cell was 10,000) in 100 μL of EGM-2 medium. After five days in suspension culture, the spheroids were introduced into the microfluidic device. To quantify spheroid diameter, fluorescent images for day 5 were converted into 8-bit images and binarized based on a threshold automatically determined by the default setting in Fiji (http://fiji.sc.), an open access software. Photographs of the co-culture spheroids are shown in FIGS. 12A-12F, and FIG. 12G is a plot of the average diameter of 10000 cells of the co-culture spheroids on day 5.

A multicellular tumor spheroid, after five days in suspension culture, was drawn into a pipette tip from a well of the ultra-low adhesion U-bottom 96-well plate and dropped into a collagen gel (2 mg/mL collagen (Corning) prepared in Dulbecco's phosphate-buffered saline (D-PBS)). The spheroid suspended in the collagen gel was then introduced into the tumor chamber. Cell-free collagen (2 mg/mL) was injected into secondary tissue sites. Owing to the surface tension at the microposts separating the chamber from the microfluidic channels, the collagen gel filled only the tissue chambers without leaking into the microfluidic channels. After gelation of the collagen gel at 37° C. for 20 min, microfluidic channels were coated with gelatin, washed with D-PBS, and filled with EGM-2 and incubated for over 1 h to remove bubbles at the gel-medium interface. HUVECs (5×10⁶ cells/mL in the EGM-2) were then introduced into the microfluidic channels. The device was then kept at 37° C. and 5% CO₂ in an incubator for 2 hours until full adhesion of HUVECs to the channels. Devices were kept at 37° C. and 5% CO₂ in an incubator for the duration of the experiment.

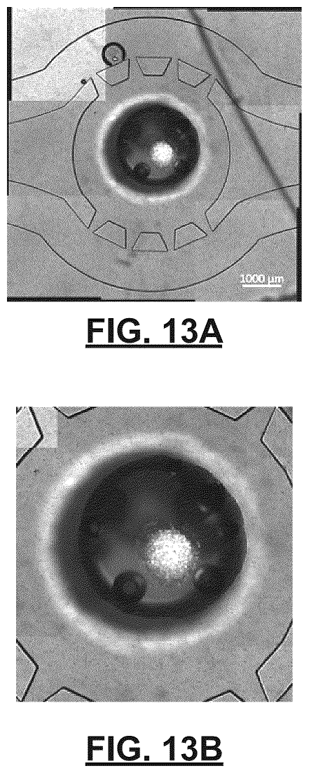

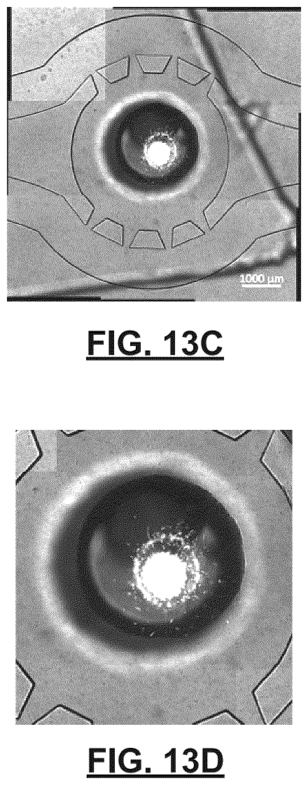

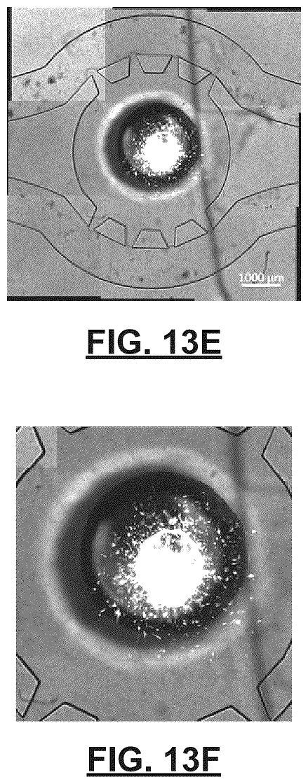

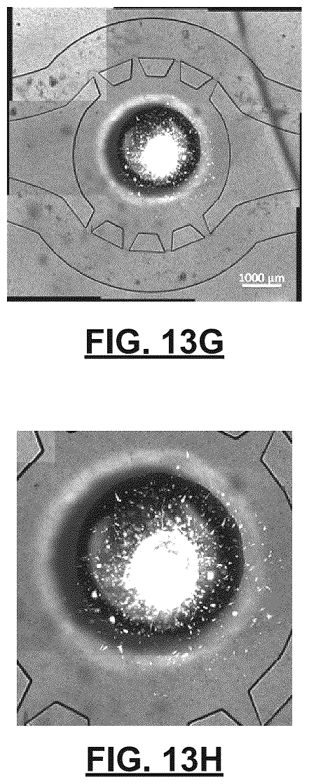

Meanwhile, the EGM-2 at the inlets of the microfluidic channels were replaced every day. After 24 hours, tumor cell invasion was observed in the tumor chamber using a Zeiss inverted microscope. To quantify the area of tumor growth, fluorescent images for days 0, 1, 3, 5, and 7 were converted into 8-bit images and binarized based on a threshold automatically determined by the default setting in Fiji (http://fiji.sc.), an open access software. The binarized image of day 0 was then subtracted from the images of days 1, 3, 5, and 7. The resulting images are shown in FIGS. 13A-H.

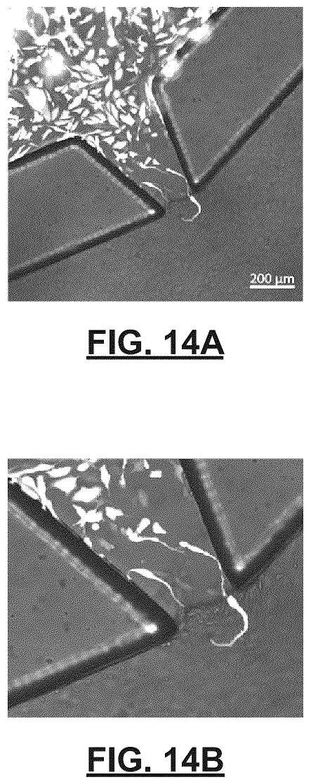

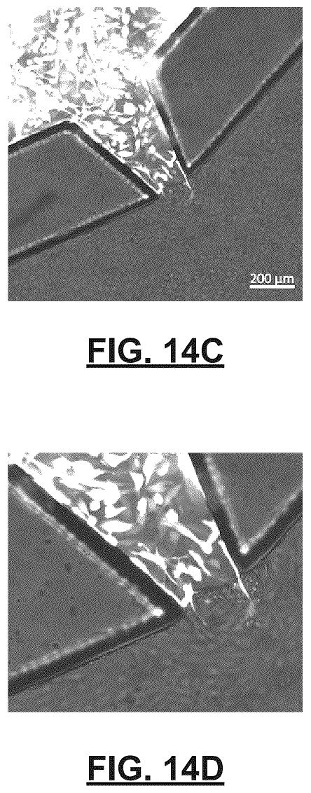

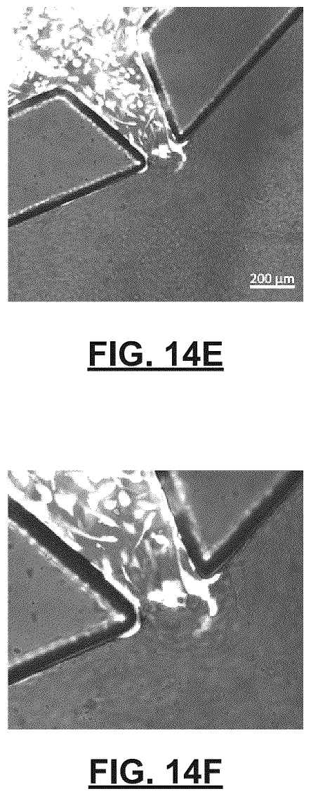

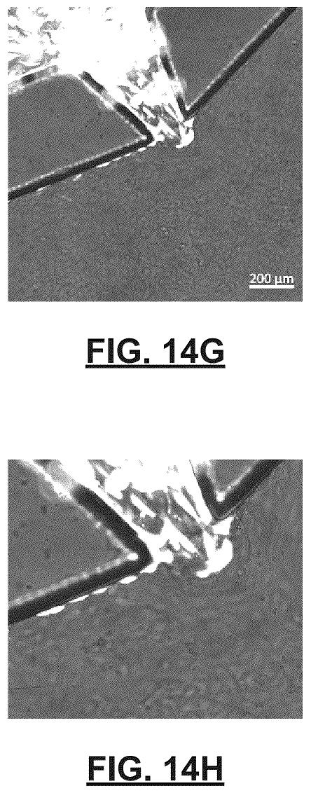

After 11 days, MDA-MB-231 cell intravasation was observed at the interface between the tumor chamber and the HUVEC-coated microfluidic channel. Fluorescent images were captured for days 11, 13, 14, and 16. The images are shown in FIGS. 14A-H.

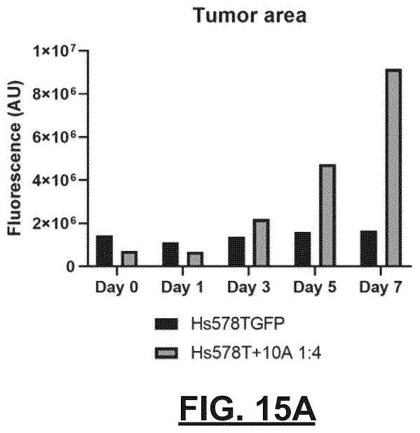

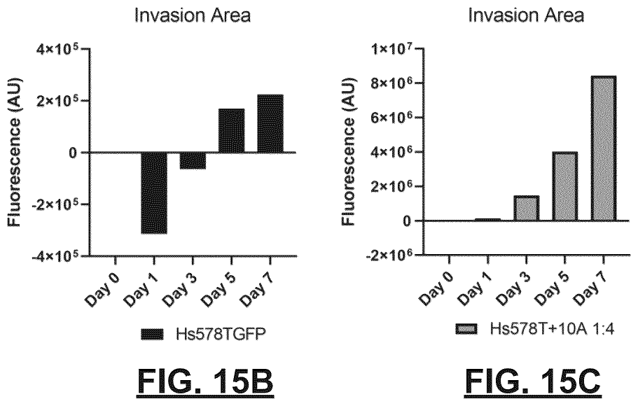

FIG. 15A is a plot of the area of the Hs578T-GFP tumor and Hs578T+10 Å 1:4 tumors over time. FIG. 15B is a plot of the invasion area of the Hs578T-GFP tumor over time, and FIG. 15C is a plot of the invasion area of the Hs578T+10 Å 1:4 tumor over time.

The following examples are described herein: Example 1: A device including a body extending away from a substrate includes a first end comprising an open-facing port configured to allow introduction of a tissue sample, and a second end of the body opposite the first end, wherein the second end forms an open outlet proximal the major surface of the substrate, and wherein at least a portion of the body comprises a tissue chamber configured to accept the tissue sample; at least one microfluidic channel on the major surface of the substrate, wherein the microfluidic channel is fluidly connected to the tissue chamber, and wherein the microfluidic channel comprises an inlet upstream of the tissue chamber and an outlet downstream of the tissue chamber; and a separation element between the tissue chamber and the at least one microfluidic channel, wherein the tissue chamber, the separation element and the microfluidic channel occupy a single layer on the substrate.

Example 2: The device of example 1, wherein the separation element comprises an arrangement of cantilevered posts extending into the primary tissue chamber from an inner surface of a wall thereof.

Example 3: The device of example 2, wherein the posts have cross-sectional shapes selected from at least one of a rectangular shape, a triangular shape, a trapezoidal shape, or a combination thereof.

Example 4: The device of example 3, wherein the posts are separated by a post-to-post spacing of about 1 micron to about 500 microns.

Example 5: The device of any of examples 1 through 4, wherein the body has a height of about 1 mm to about 5 mm above the major surface of the substrate.

Example 6: The device of any of examples 2 through 5, wherein the at least one microfluidic channel extends laterally from the body.

Example 7: The device of example 6, wherein the device comprises a plurality of microfluidic channels arranged on opposed sides of the body.

Example 8: The device of any of examples 1 through 7, wherein the tissue chamber is a primary tissue chamber, and wherein the device further comprises at least one secondary tissue chamber fluidly connected to the primary tissue chamber via the microfluidic channel.

Example 9: The device of example 8, wherein the secondary tissue chamber comprises a first portion on a first side of the microfluidic channel and a second portion on a second side of the microfluidic channel opposite the first side thereof.

Example 10: The device of any of examples 1 through 9, wherein at least one of the body, the microfluidic channel, or the separation element are formed from a plurality of overlying layers of polymeric filaments.

Example 11: The device of example 10, wherein the polymeric filaments comprise an elastic polymeric material chosen from any of silicones, (meth)acrylates, polystyrene, biodegradable polymers, hydrogels, PEGDA, biocompatible polymers, thiolenes, and mixtures and combinations thereof.

Example 12: The device of any of examples 1 through 11, wherein the body comprises an arcuate wall portion and a linear wall portion, and wherein the separation element comprises a plurality of posts arranged adjacent to the linear wall portion and extend away from the major surface of the substrate.

Example 13: The device of example 12, wherein the microfluidic channel extends laterally from the body.

Example 14: The device of any of examples 12 and 13, wherein the posts have a generally cylindrical shape.

Example 15: The device of any of examples 12 through 14, wherein the tissue chamber is a primary tissue chamber, and wherein the device further comprises at least one secondary tissue chamber fluidly connected to the primary tissue chamber.

Example 16: The device of example 15, further comprising a plurality of secondary tissue chambers fluidly connected to the primary tissue chamber.

Example 17: The device of example 15, wherein the secondary tissue chambers are arranged in series with the primary tissue chamber.

Example 18: The device of example 15, wherein the secondary tissue chambers are arranged in parallel with the primary tissue chamber.

Example 19: The device of any of examples 12 through 18, wherein at least one of the body, the microfluidic channel, or the posts are formed from a plurality of overlying layers of polymeric filaments.

Example 20: The device of example 19, wherein the polymeric filaments comprise an elastic polymeric material chosen from any of silicones, (meth)acrylates, polystyrene, biodegradable polymers, hydrogels, PEGDA, biocompatible polymers, thiolenes, and mixtures and combinations thereof.

Example 21: The device of any of examples 1 through 20, wherein the substrate comprises a biocompatible material.

Example 22: The device of example 21, wherein the substrate is substantially transparent to visible light.

Example 23: The device of any of examples 21 and 22, wherein the biocompatible material is chosen from glass and polymeric materials.

Example 24: The device of any of examples 1 through 23, wherein the at least one microfluidic channel comprises branches.

Example 25: The device of any of examples 1 through 24, wherein the tissue chamber is a primary tissue chamber, and wherein the device comprises a plurality of primary tissue chambers comprising the tissue chamber.

Example 26: The device of any of examples 1 through 25, further comprising a media inlet and a media outlet fluidly connected to the at least one microfluidic channel.

Example 27: The device of example 26, further comprising a pump connected to the media inlet.

Example 28: The device of any of examples 1 through 27, wherein the tissue chamber is configured to accept a tissue sample having a diameter of at least 500 microns.

Example 29: The device of any of examples 1 through 28, wherein the tissue sample is chosen from multicellular tumor spheroids derived from established cell lines, engineered animal models, patient-derived xenografts, or primary tumor biopsies.

Example 30: A device includes a substrate with a major surface; a body extending away from the major surface of the substrate, wherein the body comprises: a first end comprising an open-facing port configured to allow introduction into the tissue chamber of a tissue sample chosen from multicellular tumor spheroids derived from established cell lines, engineered animal models, patient-derived xenografts, or primary tumor biopsies, wherein the tissue sample has a diameter of greater than about 500 microns, and an open second end proximal the major surface of the substrate; a primary tissue chamber between the first end of the body and the second end of the body; at least one microfluidic channel on the major surface of the substrate, wherein the microfluidic channel underlies the body, and wherein the microfluidic channel comprises an inlet upstream of the tissue chamber and an outlet downstream of the tissue chamber; a membrane between the primary tissue chamber and the at least one microfluidic channel; and a secondary tissue chamber fluidly connected to the primary tissue chamber, wherein the secondary tissue chamber comprises at least one portion on a first side of the microfluidic channel and a second portion on a second side of the microfluidic channel.

Example 31: The device of example 30, wherein at least a portion of at least one of the body and the microfluidic channel are formed from overlying layers of polymeric filaments.

Example 32: The device of any of examples 30 and 31, wherein the membrane comprises a porous polymer.

Example 33: The device of example 32, wherein the polymer comprises poly dimethylsiloxane (PDMS).

Example 34: The device of any of examples 32 and 33, wherein the substrate comprises a transparent biocompatible material chosen from glass and polymers.

Example 35: A device includes a microfluidic channel; a body with a first compartment on a first side of the microfluidic channel and a second compartment on a second side of the microfluidic channel, wherein the first compartment comprises a tissue chamber with an open port configured to allow introduction of a tissue sample chosen from multicellular tumor spheroids derived from established cell lines, engineered animal models, patient-derived xenografts, or primary tumor biopsies, wherein the tissue sample has a diameter of greater than about 500 microns, and wherein the first compartment and the second compartment are fluidly connected to the microfluidic channel; a media reservoir comprising a media inlet and a media outlet, wherein the media inlet and the media outlet are fluidly connected to the microfluidic channel; and a removable plug in the microfluidic channel, wherein the removable plug controls flow of a media composition from the media reservoir into the microfluidic channel.

Example 36 The device of example 35, wherein at least one of the body or the media reservoir is formed from a plurality of overlying polymeric filaments:

Example 37: The device of any of examples 35 and 36, wherein the device further comprises at least one secondary tissue chamber fluidly connected to the primary tissue chamber.

Example 38: The device of any of examples 35 through 37, wherein the plug is chosen from metal pins and polymeric pins.

Example 39: The device of any of examples 35 through 38, further comprising an extracellular matrix material in the tissue chamber.

Example 40: An in vitro assay system includes a substrate; a body extending away from a major surface of the substrate, wherein the body comprises an open first end sized to allow introduction of a tissue sample chosen from multicellular tumor spheroids derived from established cell lines, engineered animal models, patient-derived xenografts, or primary tumor biopsies, wherein the tissue sample has a diameter of greater than about 500 microns and an open second end opposite the first end, wherein the body comprises therein a tissue chamber, an extracellular matrix material in at least a portion of the tissue chamber; at least one microfluidic channel on the major surface of the substrate, wherein the at least one microfluidic channel comprises a media inlet upstream of the tissue chamber and a media outlet downstream of the tissue chamber; a separation element between the tissue chamber and the microfluidic channel, wherein the separation element is configured to retain the extracellular matrix material in the tissue chamber, and wherein the separation element comprises at least one passage containing the extracellular matrix material; and an interface region downstream of the at least one passage in the separation element, wherein the interface region provides fluid communication between the extracellular matrix material and a media composition in the at least one microfluidic channel.

Example 41: The in vitro assay system of example 40, and wherein the microfluidic channel has an inner diameter of about 200 microns to about 1 mm.

Example 42: The in vitro assay system of any of examples 40 and 41, wherein the microfluidic channel comprises an endothelial cell layer on an inner surface thereof.

Example 43: The in vitro assay system of any of examples 40 through 42, wherein the system further comprises a pump connected to at least one of the media inlet or the media outlet.

Example 44: The in vitro assay system of example 43, wherein the pump is configured to provide continuous perfusion of the tissue chamber.

Example 45: The in vitro assay system of any of examples 43 and 44, wherein the pump is configured to provide pulsed perfusion of the tissue chamber.

Example 46: The in vitro assay system of any of examples 43 through 45, wherein the pump is configured to recirculate the media composition from the media outlet to the media inlet.

Example 47: The in vitro assay system of any of examples 40 through 46, wherein the media composition comprises a biological agent chosen from a peptide fragment, a nuclease, a nucleic acid encoding a nuclease, oligo nucleotide, a protein, peptide a DNA editing template, guide RNA, a therapeutic agent, a plasmid DNA encoding protein, siRNA, monoclonal antibodies, Cas9 mRNA, and mixtures and combinations thereof.

Example 48: The in vitro assay system of any of examples 40 through 47, wherein the at least one microfluidic channel comprises at least one branch.

Example 49: The in vitro assay system of any of examples 40 through 48, further comprising at least one secondary tissue chamber fluidly connected to the at least one microfluidic channel.

Example 50: The in vitro assay system of example 49, wherein the secondary tissue chamber comprises an extracellular matrix material.

Example 51: The in vitro assay system of any of examples 49 and 50, wherein the secondary tissue chamber is configured to house a cellular body chosen from tumor cells, aggregates, organoids, and tumor spheroids.

Example 52: The in vitro assay system of any of examples 40 through 51, wherein the system further comprises a plurality of secondary tissue chambers fluidly connected to the at least one microfluidic channel.

Example 53: The in vitro assay system of example 52, wherein the secondary tissue chambers are arranged in series with the primary tissue chamber.

Example 54: The in vitro assay system of any of examples 52 and 53, wherein the secondary tissue chambers are arranged in parallel with the primary tissue chamber.

Example 55: The in vitro assay system of any of examples 40 through 54, wherein the at least one microfluidic channel extends laterally from the primary tissue chamber.

Example 56: The in vitro assay system of example 55, wherein the system comprises a plurality of microfluidic channels arranged on opposed sides of the primary tissue chamber.

Example 57: The in vitro assay system of any of examples 40 through 56, wherein the at least one microfluidic channel underlies the primary tissue chamber.

Example 58: The in vitro assay system of any of examples 40 through 57, wherein the system comprises a syringe connected to the media inlet or outlet.

Example 59: The in vitro assay system of any of examples 40 through 58, wherein the substrate comprises glass, metal, or a polymeric material.

Example 60: The in vitro assay system of any of examples 40 through 59, wherein at least one of the body, the microfluidic channel, or the posts comprises a plurality of overlying layers of elongate polymeric filaments.

Example 61: The in vitro assay system of example 60, wherein the polymeric filaments comprise an elastic polymeric material chosen from any of silicones, (meth)acrylates, polystyrene, biodegradable polymers, hydrogels, PEGDA, biocompatible polymers, thiolenes, and mixtures and combinations thereof.

Example 62: The in vitro assay system of example 61, wherein the polymeric filaments comprise a silicone compound.

Example 63: The in vitro assay system of any of examples 40 through 62, wherein the body has a diameter of at least 1 mm and a height of at least 5 mm above the major surface of the substrate.

Example 64: A method for making a three-dimensional in vitro assay includes extruding through a nozzle an elongate polymeric base filament in a pattern on a surface of a flexible substrate; moving the nozzle to stepwise extrude and stack a plurality of polymeric filaments onto the base filament such that each of the polymeric filaments extruded onto the base filament contact one another along their lengths to form a device includes a body extending away from a major surface of the substrate, the body comprising an open top port and an open bottom opposite the top port, wherein the open top port is sized to allow introduction into the body of a tissue sample chosen from multicellular tumor spheroids derived from established cell lines, engineered animal models, patient-derived xenografts, or primary tumor biopsies, wherein the tissue sample has a diameter of greater than about 500 microns, a primary tissue chamber in the body between the open top port and the open bottom thereof, at least one microfluidic channel on the major surface of the substrate wherein the at least one microfluidic channel is fluidly connected to the primary tissue chamber; and a separation element between the primary tissue chamber and the microfluidic channel, wherein the separation element is configured to retain an extracellular matrix material in the primary tissue chamber; and at least partially curing polymeric filaments to form a self-supporting arrangement of structures on the surface of the substrate.

Example 65: The method of example 64, wherein the microfluidic channel has an inner diameter of about 200 microns to about 1 mm.

Example 66: The method of any of examples 64 and 65, wherein the body comprises a circumferential wall, and wherein the separation element comprises an arrangement of cantilevered posts extending into the tissue chamber from an inner surface of the circumferential wall thereof.

Example 67: The method of example 66, wherein the posts have a cross-sectional shape chosen from rectangular, triangular, trapezoidal, and combinations thereof.

Example 68: The method of any of examples 64 through 67, wherein the at least one microfluidic channel extends laterally from the tissue chamber.

Example 69: The method of any of examples 64 through 68, wherein the device comprises a plurality of microfluidic channels arranged on opposed sides of the body.

Example 70: The method of any of examples 64 through 69, further comprising extruding the filaments to form at least one secondary tissue chamber fluidly connected to the tissue chamber via the microfluidic channel.

Example 71: The method of any of examples 64 through 70, wherein the body has a diameter of at least 1 mm and a height of at least 5 mm above the major surface of the substrate.

Example 72: The method of any of examples 64 through 71, wherein the body comprises an wall with an arcuate portion, and wherein the posts are arranged opposite the arcuate portion of the wall, and wherein the posts extend away from the major surface of the substrate.

Example 73: The method of example 72, wherein the microfluidic channel extends laterally from the tissue chamber.

Example 74: The method of any of examples 72 and 73, wherein the posts have a generally cylindrical shape.

Example 75: The method of any of examples 64 through 74, wherein the polymeric filaments comprise an elastic polymeric material chosen from any of silicones, (meth)acrylates, polystyrene, biodegradable polymers, hydrogels, PEGDA, biocompatible polymers, thiolenes, and mixtures and combinations thereof.

Example 76: The method of any of examples 64 through 75, further comprising forming with the filaments a media inlet upstream of the body and a media outlet downstream of the body.

Example 77: The method of any of examples 64 through 76, wherein the substrate is non-planar.

Example 78: The method of any of examples 64 through 77, wherein the filaments further comprise ceramic particles, metal particles, and mixtures and combinations thereof.

Example 79: The method of any of examples 64 through 78, wherein the filaments have a cross-sectional diameter of about 100 nm to about 500 μm.

Example 80: The method of any of examples 64 through 79, wherein the at least one microfluidic channel has a burst pressure of greater than about 25 kPa.

Example 81: The method of any of examples 64 through 80, further comprising curing the filaments with ultraviolet (UV) light.

Example 82: A method for modeling tumor development includes in a tumor microenvironment includes a substrate; a body on a major surface of the substrate, the body comprising an open first end and an open second end opposite the first end, wherein the body comprises a tissue chamber between the open first end and the open second end thereof, and wherein the tissue chamber comprises an extracellular matrix material; at least one microfluidic channel on the major surface of the substrate, wherein the microfluidic channel comprises an inner surface with a layer of endothelial cells, and wherein the microfluidic channel has an inner diameter of about 200 microns to about 1 mm; and a separation element between the tissue chamber and the microfluidic channel, wherein the separation element is configured to retain a tissue sample in the well, and wherein the separation element comprises at least one passage containing the extracellular matrix material; and a media inlet upstream of the tissue chamber and a media outlet downstream of the tissue chamber; inserting the tissue sample into the tissue chamber, wherein the tissue sample is chosen from multicellular tumor spheroids derived from established cell lines, engineered animal models, patient-derived xenografts, or primary tumor biopsies, and wherein the tissue sample has a diameter of greater than about 500 microns; injecting a media composition into the media inlet to perfuse the tissue chamber; and forming an interface region downstream of the separation element, wherein the interface region provides fluid communication between extracellular matrix material and the media composition in the at least one microfluidic channel.

Example 83: The method of example 82, wherein the tissue sample is inserted into the tissue chamber with a biopsy punch.

Example 84: The method of any of examples 82 and 83, wherein the extracellular matrix material comprises collagen.

Example 85: The method of any of examples 82 through 84, wherein the tumor spheroid comprises a vascular structure.

Example 86: The method of any of examples 82 through 85, wherein the media composition comprises a biological agent chosen from a peptide fragment, a nuclease, a nucleic acid encoding a nuclease, oligo nucleotide, a protein, peptide a DNA editing template, guide RNA, a therapeutic agent, a plasmid DNA encoding protein, siRNA, monoclonal antibodies, Cas9 mRNA, and mixtures and combinations thereof.

Example 87: The method of example 86, wherein the therapeutic agent comprises at least one drug.

Example 88: The method of any of examples 82 through 87, wherein the media composition is applied to the at least one microfluidic channel by a pump.

Example 89: The method of example 88, wherein the media composition is applied continuously.

Example 90: The method of any of examples 88 and 89, wherein the media composition is applied in a pulsed flow pattern.

Example 91: The method of any of examples 88 through 90, wherein the tumor microenvironment further comprises a media outlet, and wherein the pump recirculates the media composition from the media outlet to the media inlet.

Example 92: The method of any of examples 82 through 91, wherein the tumor microenvironment further comprises at least one secondary tissue chamber.

Example 93: The method of example 92, further comprising recirculating the media composition from the outlet into the media inlet.

Example 94: The method of any of examples 82 through 93, wherein at least one of the tissue chamber, the at least one microfluidic channel, and the posts comprises overlying polymeric filaments.

Example 95: The method of example 94, wherein the polymeric filaments are extruded onto the major surface of the substrate.

Example 96: A method includes three-dimensionally (3D) printing a device includes a body extending away from the substrate, wherein a first end of the body comprises an open-facing port configured to allow introduction of a tissue sample chosen from multicellular tumor spheroids derived from established cell lines, engineered animal models, patient-derived xenografts, or primary tumor biopsies, wherein the tissue sample has a diameter of greater than about 500 microns, and a second end of the body opposite the first end forms an open outlet proximal the major surface of the substrate, and wherein at least a portion of the body comprises therein a primary tissue chamber; at least one microfluidic channel on the major surface of the substrate, wherein the microfluidic channel is fluidly connected to the tissue chamber, and wherein the microfluidic channel comprises an inlet upstream of the tissue chamber and an outlet downstream of the tissue chamber; and a separation element between the primary tissue chamber and the at least one microfluidic channel; and wherein the tissue chamber, the separation element and the microfluidic channel occupy a single layer on the substrate.

Example 97: The method of example 96, wherein 3D printing comprises extruding overlying polymeric filaments onto the substrate to form at least one of the body, the posts, or the microfluidic channel.

Example 98: The method of any of examples 96 and 97, wherein 3D printing comprises extruding overlying polymeric filaments onto the substrate to form the body, the separation element, or the microfluidic channel.

Various examples have been described. These and other examples are within the scope of the following claims.

1. A device comprising:
a body extending away from a substrate, the body comprising:
a first end comprising an open-facing port configured to allow introduction of a tissue sample, and
a second end of the body opposite the first end, wherein the second end forms an open outlet proximal the major surface of the substrate, and wherein at least a portion of the body comprises a tissue chamber configured to accept the tissue sample;
at least one microfluidic channel on the major surface of the substrate, wherein the microfluidic channel is fluidly connected to the tissue chamber, and wherein the microfluidic channel comprises an inlet upstream of the tissue chamber and an outlet downstream of the tissue chamber; and
a separation element between the tissue chamber and the at least one microfluidic channel, wherein the tissue chamber, the separation element and the microfluidic channel occupy a single layer on the substrate.

2. The device of claim 1, wherein the separation element comprises an arrangement of cantilevered posts extending into the primary tissue chamber from an inner surface of a wall thereof.

3. The device of claim 2, wherein the posts have cross-sectional shapes selected from at least one of a rectangular shape, a triangular shape, a trapezoidal shape, or a combination thereof.

4. The device of claim 3, wherein the posts are separated by a post-to-post spacing of about 1 micron to about 500 microns.

5. The device of claim 1, wherein the body has a height of about 1 mm to about 5 mm above the major surface of the substrate.

6. The device of claim 1, wherein the device comprises a plurality of microfluidic channels arranged on opposite sides of the body.

7. The device of claim 1, wherein the tissue chamber is a primary tissue chamber, and wherein the device further comprises at least one secondary tissue chamber fluidly connected to the primary tissue chamber via the microfluidic channel.

8. The device of claim 7, wherein the secondary tissue chamber comprises a first portion on a first side of the microfluidic channel and a second portion on a second side of the microfluidic channel opposite the first side thereof.

9. The device of claim 8, wherein the secondary tissue chambers are arranged in series with the primary tissue chamber.

10. The device of claim 1, wherein at least one of the body, the microfluidic channel, or the separation element are formed from a plurality of overlying layers of polymeric filaments.

11. The device of claim 1, wherein the body comprises an arcuate wall portion and a linear wall portion, and wherein the separation element comprises a plurality of posts arranged adjacent to the linear wall portion and extend away from the major surface of the substrate.

12. The device of claim 1, wherein the substrate comprises a biocompatible material.

13. The device of claim 1, am, wherein the at least one microfluidic channel comprises branches.

14. The device of claim 1, further comprising a media inlet and a media outlet fluidly connected to the at least one microfluidic channel.

15. The device of claim 1, wherein the tissue chamber is configured to accept a tissue sample having a diameter of at least 100 microns.

16. The device of claim 1, wherein the tissue sample is chosen from multicellular tumor spheroids derived from established cell lines, engineered animal models, patient-derived xenografts, or primary tumor biopsies.

17. A method comprising:
three-dimensionally (3D) printing a device comprising:
a body extending away from a substrate, wherein a first end of the body comprises an open-facing port configured to allow introduction of a tissue sample chosen from multicellular tumor spheroids derived from established cell lines, engineered animal models, patient-derived xenografts, or primary tumor biopsies, wherein the tissue sample has a diameter of greater than about 500 microns, wherein a second end of the body opposite the first end forms an open outlet proximal the major surface of the substrate, and wherein at least a portion of the body comprises therein a primary tissue chamber;
at least one microfluidic channel on the major surface of the substrate, wherein the microfluidic channel is fluidly connected to the tissue chamber, and wherein the microfluidic channel comprises an inlet upstream of the tissue chamber and an outlet downstream of the tissue chamber; and
a separation element between the primary tissue chamber and the at least one microfluidic channel; and wherein the tissue chamber, the separation element and the microfluidic channel occupy a single layer on the substrate.

18. The method of claim 17, wherein 3D printing comprises extruding overlying polymeric filaments onto the substrate to form at least one of the body, the posts, or the microfluidic channel.

19. The method of claim 17, wherein 3D printing comprises extruding overlying polymeric filaments onto the substrate to form at least one of the body, the separation element, or the microfluidic channel.

20. An in vitro assay system comprising:
a substrate;
a body extending away from a major surface of the substrate, wherein the body comprises an open first end sized to allow introduction of a tissue sample chosen from multicellular tumor spheroids derived from established cell lines, engineered animal models, patient-derived xenografts, or primary tumor biopsies, wherein the tissue sample has a diameter of greater than about 500 microns and an open second end opposite the first end, wherein the body comprises therein a tissue chamber, an extracellular matrix material in at least a portion of the tissue chamber;
at least one microfluidic channel on the major surface of the substrate, wherein the at least one microfluidic channel comprises a media inlet upstream of the tissue chamber and a media outlet downstream of the tissue chamber;
a separation element between the tissue chamber and the microfluidic channel, wherein the separation element is configured to retain the extracellular matrix material in the tissue chamber, and wherein the separation element comprises at least one passage containing the extracellular matrix material; and
an interface region downstream of the at least one passage in the separation element, wherein the interface region provides fluid communication between the extracellular matrix material and a media composition in the at least one microfluidic channel.
